(19) United States
(12) Patent Application Publication
(10) Pub. No.: US 2022/0233365 A1
(43) Pub. Date: Jul. 28, 2022

(54) ABSORBENT ARTICLE WITH REAR POCKET DESIGN

(71) Applicant: Kimberly-Clark Worldwide, Inc., Neenah, WI (US)

(72) Inventors: Carlos E. Roldan-Posada, Medellin (CO); Susan K. Hanson, Greenville, WI (US)

(21) Appl. No.: 16/471,687

(22) PCT Filed: Dec. 30, 2016
(86) PCT No.: PCT/US2016/069427

§ 371 Date: Jun. 20, 2019

(51) Int. Cl. A61F 13/495; A61F 13/15; A61F 13/49

(52) U.S. Cl. CPC A61F 13/495; A61F 13/15723; A61F 2013/15861; A61F 13/49011; A61F 2013/4951; A61F 13/15764

(57) ABSTRACT

Absorbent articles comprising connecting portions and methods of manufacture are disclosed. In an embodiment an absorbent article may comprise a chassis having a front half section, a rear half section, a body facing surface, and a garment facing surface. The article further comprises an absorbent body and a of containment flaps attached to the body facing surface of the chassis, each of the pair of containment flaps having a flap distal edge. The article may further comprise a connecting portion in the rear half section where the containment flaps and connecting portion define an opening to the body facing surface. The opening may be defined by a pair of laterally opposed edges having a minimum separation distance within the rear end section of between about one-quarter and about three-quarters of a distance between the flap distal edges of the containment flaps.

### TECHNICAL FIELD

The present disclosure relates to absorbent articles.

### BACKGROUND OF THE DISCLOSURE

A primary function of personal care absorbent articles is to absorb and retain body exudates such as urine, fecal material, blood, and menses with additional desired attributes including low leakage of the exudates from the absorbent article and a dry feel to the wearer of the absorbent article. By preventing leakage of the exudates from the absorbent article, the absorbent article intends to prevent the body exudates from soiling or contaminating a wearer's or caregiver's clothing or other articles, such as bedding, that can come in contact with the wearer.

Another important emphasis of some absorbent articles is to draw and retain exudates away from the wearer's skin and otherwise reduce exposure of the wearer's skin to the exudates. Drawing away and reducing skin exposure to the exudates can help to keep the skin dry and to ensure that there is minimal exposure of irritants in the exudates to the skin. These functions can help to promote and maintain healthy skin for the wearer. Absorbent articles with enhanced abilities to draw away and store exudates and/or reduce skin exposure to exudates are continually desired to help ensure that the absorbent article wearer's skin remains healthy.

### SUMMARY OF THE DISCLOSURE

The absorbent articles disclosed herein are designed to reduce exposure of a wearer's skin to exudates after a bowel movement (BM). In general, the absorbent articles disclosed herein are constructed so as to cover a large extent of a wearer's skin, particularly in a rear end section of the absorbent articles.

In a first embodiment, an absorbent article can comprise a chassis extending in a longitudinal direction and a lateral direction, the chassis having a front half section with a front waist region including a front waist edge, a rear half section having a rear waist region including a rear waist edge, a body facing surface, and a garment facing surface, and an absorbent body. The article may further comprise a pair of containment flaps extending between the front waist region and the rear waist region and attached to the body facing surface of the chassis, each of the pair of containment flaps having a flap distal edge, and the pair of containment flaps further including a connecting portion in the rear half section where the containment flaps and connecting portion define an opening to the body facing surface in the rear end section. Additionally, the opening may be defined by a pair of laterally opposed edges having a minimum separation distance within the rear end section of between about one-quarter and about three-quarters of a distance between the flap distal edges of the containment flaps.

In a second embodiment, the pair of laterally opposed edges of the absorbent article of the first embodiment may have a minimum separation distance between about one-third and about two-thirds of the distance between the flap distal edges of the containment flaps.

In a third embodiment, the opening of any of the first or second embodiments may be further defined by a laterally extending back edge, and the lateral extent of the laterally extending back edge may comprise the minimum separation distance.

In a fourth embodiment, each of the laterally opposed edges of any of the first through third embodiments may form an angle with the laterally extending back edge of between about 60 degrees to about 85 degrees.

In a fifth embodiment, each of the laterally opposed edges of any of the first through fourth embodiments may form an angle with the laterally extending back edge of between about 65 degrees to about 75 degrees.

In a sixth embodiment, the connecting portion of any of the first through fifth embodiments may comprise a web of material bonded to each of the pair of containment flaps.

In a seventh embodiment, an absorbent article may comprise a chassis having a front half section with a front waist region including a front waist edge, a rear half section having a rear waist region including a rear waist edge, a body facing surface, and a garment facing surface, an absorbent body, and a pair of containment flaps extending between the front waist region and the rear waist region and attached to the body facing surface of the chassis, each of the pair of containment flaps having a flap distal edge. The article may further comprise a shaped patch bonded to the pair of containment flaps and the body facing surface of the absorbent article within the rear half section of the chassis. Additionally, the shaped patch may comprise a tongue portion, a base portion, and leg portions, and the leg portions may be separated from each other by a minimum of between about one-quarter and about three-quarters of a distance between the flap distal edges of the containment flaps.

In an eighth embodiment, the leg portions of the seventh embodiment may be separated from each other by a minimum of between about one-third and about two-thirds of a distance between the flap distal edges of the containment flaps.

In a ninth embodiment, each of the leg portions of any of the seventh or eighth embodiments may form an angle with the base portion of between about 60 degrees to about 85 degrees.

In a tenth embodiment, each of the leg portions of any of the seventh through ninth embodiments may form an angle with the base portion of between about 65 degrees to about 75 degrees.

In an eleventh embodiment, the shaped patch of any of the seventh through tenth embodiments may be positioned on top of each of the containment flaps.

In a twelfth embodiment, the shaped patch of any of the seventh through eleventh embodiments may be positioned between the containment flaps and the body facing surface of the chassis.

In a thirteenth embodiment, any of the seventh through twelfth embodiments may further comprise an elastic waistband, and the elastic waistband may cover at least a portion of the tongue portion of the shaped patch.

In a fourteenth embodiment, each of the leg portions of any of the seventh through thirteenth embodiments may be bonded to a respective containment flap, and wherein each of the leg portions extends laterally inward of the respective containment flap.

In a fifteenth embodiment, at least a portion of the shaped patch of any of the seventh through fourteenth embodiments may be elasticized.

In a sixteenth embodiment, a method of forming an absorbent article may comprise moving an absorbent article chassis containing an absorbent body in a machine direction, the absorbent article chassis comprising: a longitudinal direction and a lateral direction, a front half section with a front waist region including a front waist edge, a rear half section having a rear waist region including a rear waist edge, a body facing surface, and a garment facing surface. The method may further comprise moving a pair of containment flaps in the machine direction, each of the containment flaps having distal flap edges, bonding the pair of containment flaps to the absorbent article chassis at laterally opposed positions, moving a length of shaped patch material in the machine direction, and separating a shaped patch from the shaped patch material, wherein the shaped patch comprises: a tongue portion, a base portion, and leg portions. In some embodiments, the leg portions may be separated from each other by a minimum of between about one-quarter and about three-quarters of a distance between the distal flap edges of the containment flaps after the containment flaps are bonded to the absorbent article chassis. The method may finally comprise bonding the shaped patch to the pair of containment flaps and to the body facing surface.

In a seventeenth embodiment, the shaped patch of the sixteenth embodiment may be separated from the shaped patch material such that there is no waste of the shaped patch material.

In an eighteenth embodiment, the shaped patch of the sixteenth or seventeenth embodiments may bonded on top of the pair of containment flaps.

In a nineteenth embodiment, the shaped patch of any of the sixteenth through eighteenth embodiments may be bonded between the pair of containment flaps and the garment facing surface.

In a twentieth embodiment, the method of any of the sixteenth through nineteenth embodiments may further comprise bonding an elasticized waistband to the chassis of the absorbent article in the rear waist region and covering at least a portion of the shaped patch.

The above summary of the present disclosure is not intended to describe each embodiment or every implementation of the present disclosure. Advantages and attainments, together with a more complete understanding of the disclosure, will become apparent and appreciated by referring to the following detailed description and claims taken in conjunction with the accompanying drawings.

### BRIEF DESCRIPTION OF DRAWINGS

The disclosure may be more completely understood in consideration of the following detailed description of various embodiments in connection with the accompanying drawings, in which:

FIG. 1 perspective view of an absorbent article according to aspects of the present disclosure;

FIG. 1A perspective view of a different absorbent article according to aspects of the present disclosure;

FIG. 2 is a top plan view of the absorbent article of FIG. 1 in a stretched, laid flat and unattached condition and including a connecting portion, according to aspects of the present disclosure;

FIG. 2A is a top plan view of the absorbent article of FIG. 1A in a stretched, laid flat and unfastened condition and including a connecting portion, according to aspects of the present disclosure;

FIG. 3 is a cross-section of the absorbent article of FIG. 2 taken along line 3-3.

FIG. 4 is a perspective view of the absorbent article of FIG. 2 in a relaxed state showing the connecting portion;

FIG. 5 is a close-up top plan view of region A of the absorbent article depicted in FIG. 2;

FIG. 6 is a top plan view of an exemplary patch according to aspects of the present disclosure, which may be used in conjunction with the absorbent articles of the present disclosure;

FIG. 7A is a view detailing a configuration of the layers of the absorbent article of FIG. 2 as viewed from a cross-section taken along line 3-3;

FIG. 7B is a view detailing an alternative configuration of the layers of the absorbent article of FIG. 2 as viewed from a cross-section taken along line 3-3;

FIG. 8 is a top plan view of an absorbent article in a stretched, laid flat and unattached condition and including an alternative connecting portion, according to aspects of the present disclosure;

FIG. 9 is a top plan view of an absorbent article in a stretched, laid flat and unattached condition and including an alternative connecting portion, according to aspects of the present disclosure;

FIG. 10 is a perspective view of an absorbent article including a first exemplary connecting portion where the absorbent article is depicted in a configuration as it would appear if worn by a wearer;

FIG. 11 is a perspective view of an absorbent article including a second exemplary connecting portion where the absorbent article is depicted in a configuration as it would appear if worn by a wearer;

FIG. 12 is a perspective view of an absorbent article including a third exemplary connecting portion where the absorbent article is depicted in a configuration as it would appear if worn by a wearer;

FIG. 13 is a schematic perspective view of an exemplary process for forming absorbent articles of the present disclosure; and

FIG. 14 is a schematic perspective view of an alternative exemplary process for forming absorbent articles of the present disclosure.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION OF THE DISCLOSURE

The present disclosure is generally directed towards absorbent articles constructed so as to cover a large extent of a wearer's skin, particularly in a rear end section of the absorbent articles. The articles of the present disclosure may have enhanced BM flaps which are constructed so as to provide a relatively small opening to the absorbent core, and thereby provide cover to a large extent of a wearer's skin. The articles of the present disclosure may additionally or alternatively include an extra patch of material in the rear end section of the absorbent articles which connects the BM flaps and provides the large skin coverage area of the article.

Each example is provided by way of explanation and is not meant as a limitation. For example, features illustrated or described as part of one embodiment or figure can be used on another embodiment or figure to yield yet another embodiment. It is intended that the present disclosure include such modifications and variations.

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products, adult diapers and pants, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded”, “attached” or “coupled” refers herein to the joining, adhering, connecting, attaching, or the like, of two elements. Two elements will be considered bonded, attached or coupled together when they are joined, adhered, connected, attached, or the like, directly to one another or indirectly to one another, such as when each is directly bonded to intermediate elements. The bonding, attaching or coupling of one element to another can occur via continuous or intermittent bonds.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer liquids, such as, but not limited to, barrier films, filled films, breathable films, and oriented films.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, hydroentangling processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “stretch film laminate” refers herein to a laminate of an elastic film laminated to at least one outer layer of fibrous nonwoven web material. Desirably, the elastic film is air and vapor permeable and liquid impermeable and elastic in at least one and preferably two directions generally perpendicular to one another. More desirably, the elastic film is elastic in all directions of the X-Y plane of the material. In other embodiments, the elastic film can have cover layers, such as fibrous nonwoven web materials laminated to both sides of the elastic film so as so form a laminate with opposed exterior nonwoven layers, an intermediate layer of elastic film bonded to both of the exterior nonwoven layers. Further information can be found with reference to U.S. Pat. No. 7,803,244 to Siqueira et al. and U.S. Pat. No. 8,361,913 to Siqueira et al., each of which is incorporated herein in its entirety by reference.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

The term “user” or “caregiver” refers herein to one who fits an absorbent article, such as, but not limited to, a diaper, diaper pant, training pant, youth pant, incontinent product, or other absorbent article about the wearer of one of these absorbent articles. A user and a wearer can be one and the same person.

Absorbent Article:

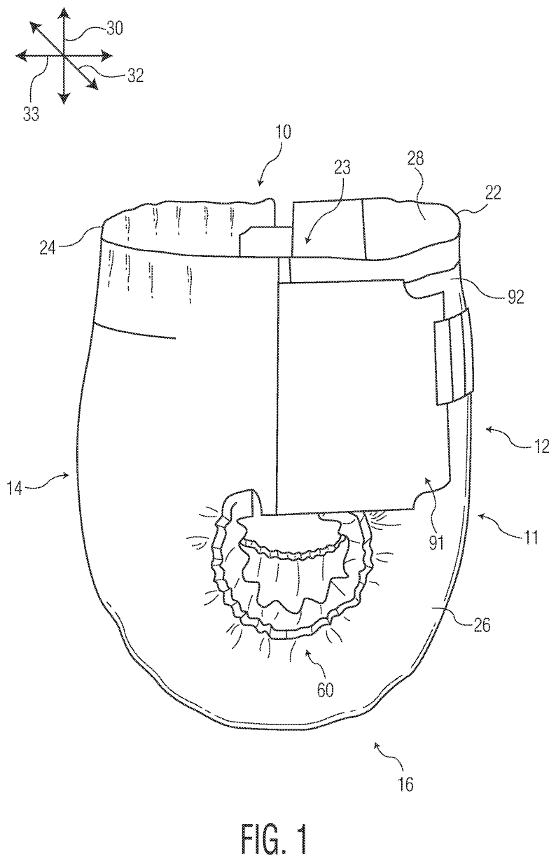

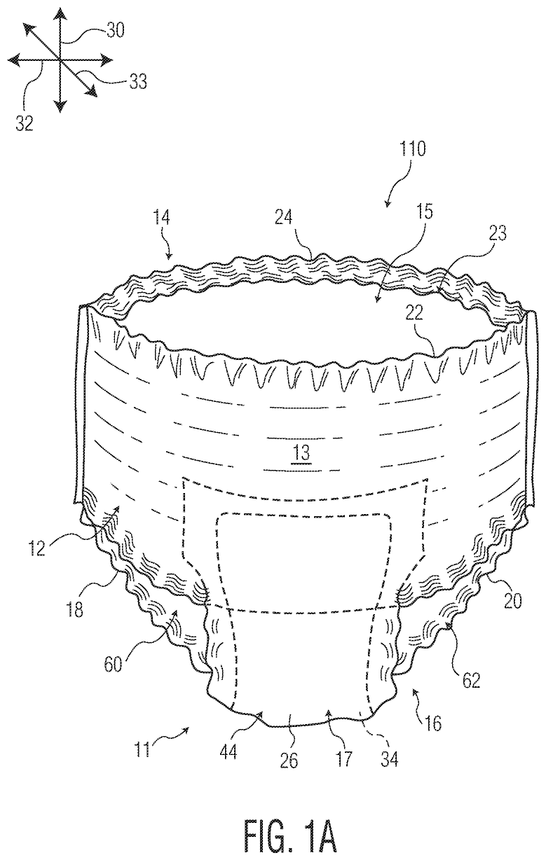

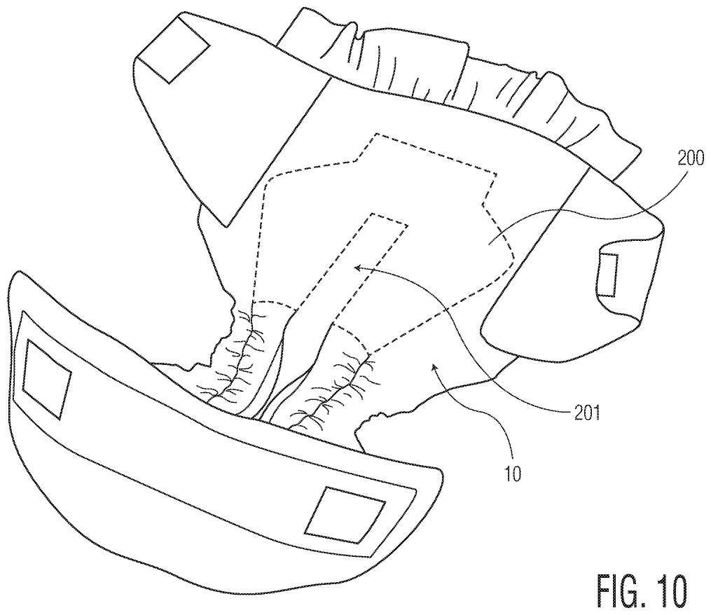

FIGS. 1 through 4 depict absorbent articles according to the present disclosure. One of the features of the present disclosure is the unique nature of the rear half section of the absorbent articles. Specifically, the absorbent of the present disclosure include a unique shape of an opening through the BM containment flaps (also referred to herein as simply “containment flaps” or “flaps” or “BM flaps”) to the bodyside liner and absorbent core of the absorbent article which provides increased protection of a wearer's skin after a BM. The shape of the opening provides adequate room for exudates from a BM to pass through the opening to contact the liner and absorbent core while still providing coverage to a large area of the wearer's skin. As the BM contacts the liner and absorbent core, the BM may spread toward the sides and the rear waist region of the absorbent article, where the skin of the wearer is protected by the BM flaps and additional material forming the unique opening. FIG. 10 depicts an exemplary configuration of an absorbent article according to the present disclosure on a wearer.

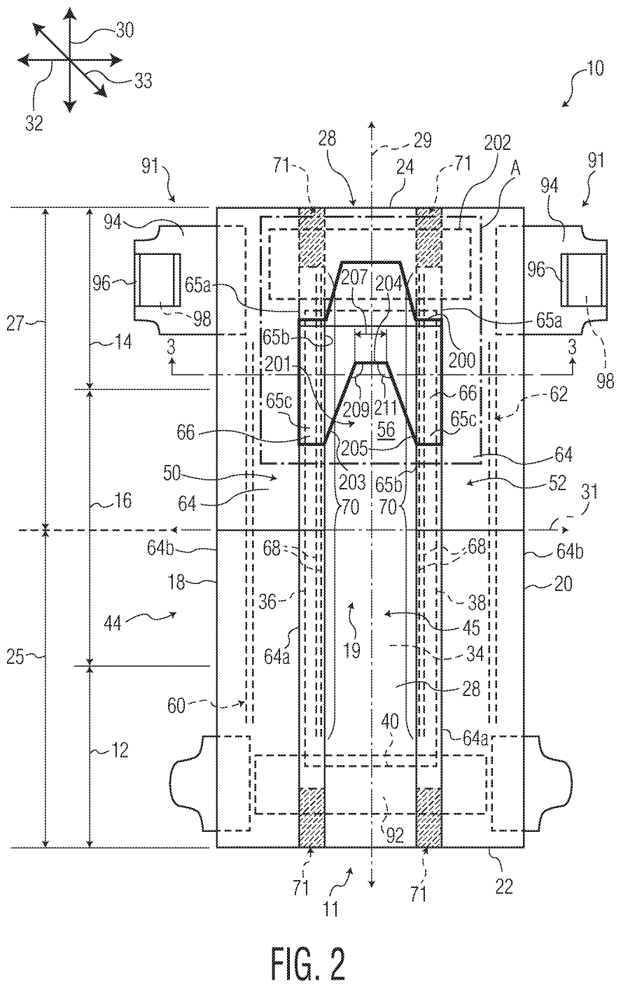

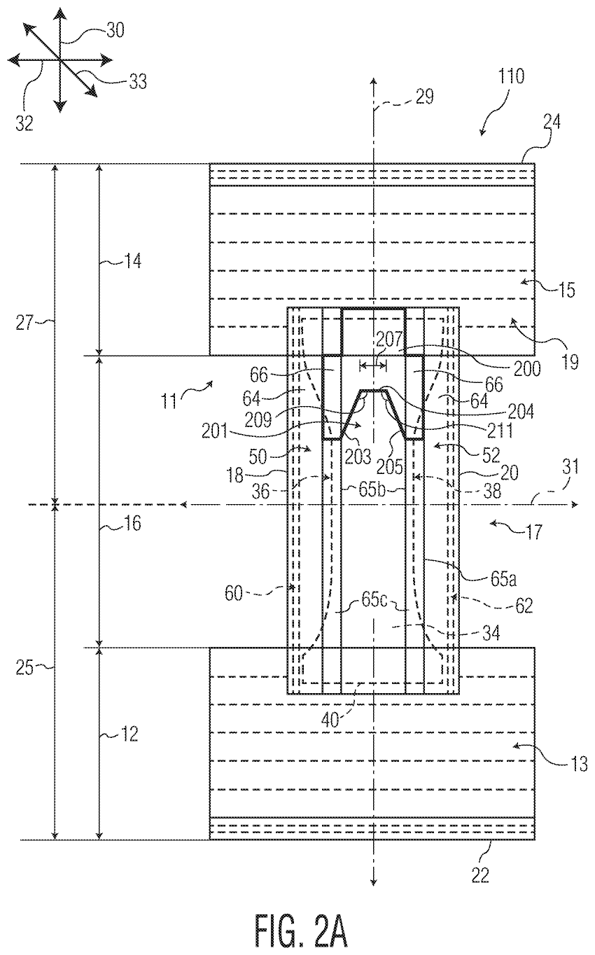

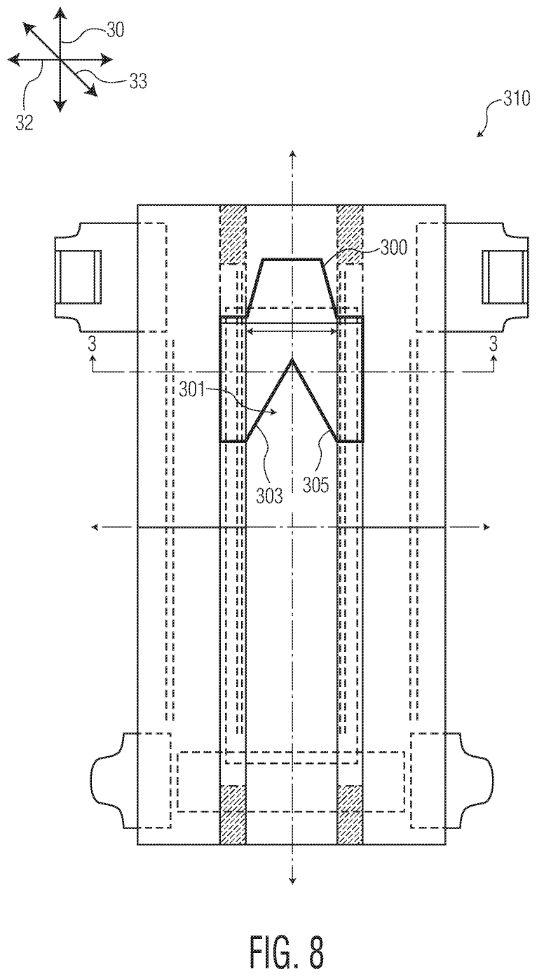

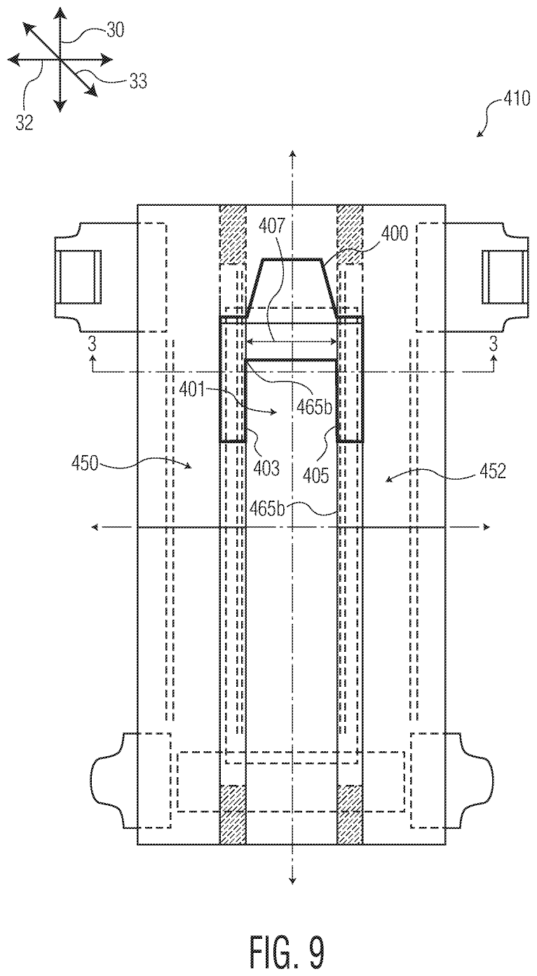

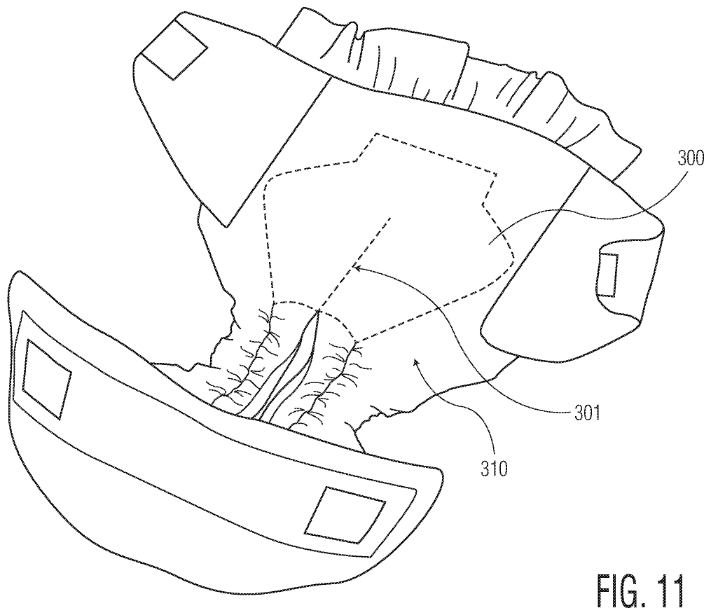

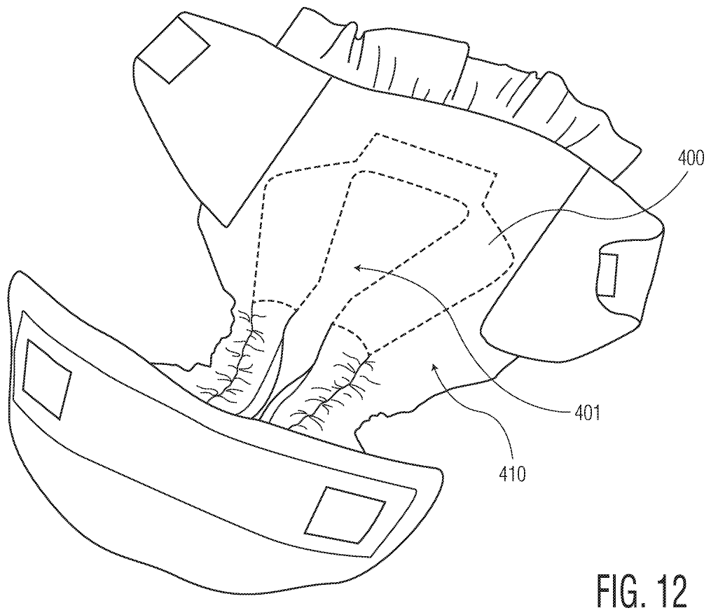

In contrast, FIGS. 8, 9, 11, and 12 depict absorbent articles with alternative configurations of an opening through the BM flaps to the liner and absorbent core and the resultant configuration of those absorbent articles on a wearer. As can be seen in FIGS. 11 and 12, the alternative configurations of the opening shapes of the absorbent articles shown in FIGS. 8 and 9 result in less optimal wear configurations of than the absorbent article described with respect to FIGS. 2-5.

Referring to FIGS. 1-4, a non-limiting illustration of an absorbent article 10 for example, a diaper, is illustrated. Other embodiments of the absorbent article 10 can include, but are not limited to, training pants, youth pants, adult incontinence garments, and feminine hygiene articles. While the embodiments and illustrations described herein may generally apply to absorbent articles manufactured in the product longitudinal direction, which is hereinafter called the machine direction manufacturing of a product, it should be noted that one of ordinary skill in the art could apply the information herein to absorbent articles manufactured in the latitudinal direction of the product, which hereinafter is called the cross direction manufacturing of a product, without departing from the spirit and scope of the disclosure. For example, the absorbent article 110 in FIGS. 1A and 2A provides an exemplary embodiment of an absorbent article 110 that can be manufactured in cross-direction manufacturing process.

The absorbent article 10 illustrated in FIGS. 1 through 4 can include a chassis 11. The absorbent article 10 can include a front waist region 12 and a rear waist region 14, and a crotch region 16 disposed between the front waist region 12 and the rear waist region 14 and interconnecting the front and rear waist regions, 12, 14, respectively. The front waist region 12 is located in the front half section 25 of the article 10, and the rear waist region 14 is located in the rear half section 27 of the article 10. In the embodiment depicted in FIGS. 1A and 2A, a three-piece construction of an absorbent article 110 is depicted where the absorbent article 110 can have a chassis 11 including a front waist panel 13 defining the front waist region 12, a rear waist panel 15 defining the rear waist region 14, and an absorbent panel 17 defining the crotch region 16 of the absorbent article 110. The absorbent panel 17 can extend between the front waist panel 13 and the rear waist panel 15. In some embodiments, the absorbent panel 17 can overlap the front waist panel 13 and the rear waist panel 15. The absorbent panel 17 can be bonded to the front waist panel 13 and the rear waist panel 15 to define a three-piece construction. However, it is contemplated that an absorbent article can be manufactured in a cross-direction without being a three-piece construction garment which is also sometimes referred to as a one-piece construction (not shown) as the front waist panel 13 and the rear waist panel 15 are integral with one another by way of commonly connected components forming the waist panel such as a bodyside liner and/or an outer cover which can envelope the absorbent panel 17 or simply cover the garment side of the absorbent panel 17.

The absorbent article 10, 110 can have a pair of longitudinal side edges 18, 20, and a pair of opposite waist edges, respectively designated front waist edge 22 and rear waist edge 24. The front waist region 12 can be contiguous with the front waist edge 22 and the rear waist region 14 can be contiguous with the rear waist edge 24. The longitudinal side edges 18, 20 can extend from the front waist edge 22 to the rear waist edge 24. The longitudinal side edges 18, 20 can extend in a direction parallel to the longitudinal direction 30 for their entire length, such as for the absorbent article 10 illustrated in FIG. 2. In other embodiments, the longitudinal side edges 18, 20 can be curved between the front waist edge 22 and the rear waist edge 24. In the absorbent article 110 of FIGS. 1A and 2A, the longitudinal side edges 18, 20 can include portions of the front waist panel 13, the absorbent panel 17, and the rear waist panel 15.

The front waist region 12 can include the portion of the absorbent article 10, 110 that, when worn, is positioned at least in part on the front of the wearer while the rear waist region 14 can include the portion of the absorbent article 10, 110 that, when worn, is positioned at least in part on the back of the wearer. The crotch region 16 of the absorbent article 10, 110 can include the portion of the absorbent article 10, 110 that, when worn, is positioned between the legs of the wearer and can partially cover the lower torso of the wearer. Dimensionally, the front waist region 12 can be defined as the front one-third of the overall longitudinal dimension of the absorbent article 10 measuring from the front waist edge 22 to the rear waist edge 24. The rear waist region 14 can be defined as the rear one-third of the longitudinal dimension of the absorbent article 10 and the crotch region 16 is the middle one-third of the longitudinal dimension of the overall absorbent article 10. The waist edges, 22 and 24, of the absorbent article 10, 110 are configured to encircle the waist of the wearer and together define a central waist opening 23 (as labeled in FIG. 1 and FIG. 1A) for the waist of the wearer. Portions of the longitudinal side edges 18, 20 in the crotch region 16 can generally define leg openings for the legs of the wearer when the absorbent article 10, 110 is worn.

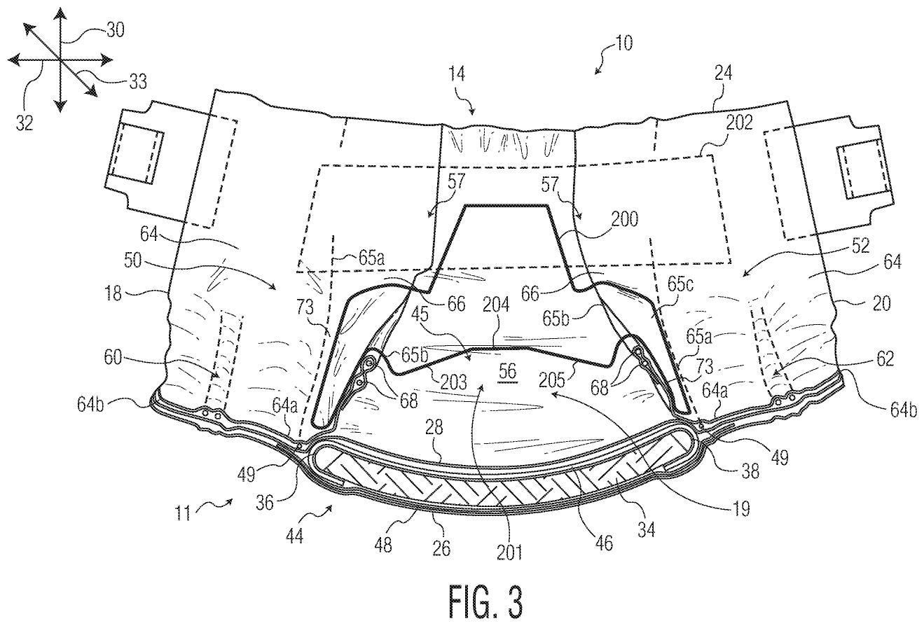

The absorbent article 10, 110 can include an outer cover 26 and a bodyside liner 28. The outer cover 26 and the bodyside liner 28 can form a portion of the chassis 11 where the outer cover is disposed at the garment facing surface of the chassis (not shown; opposite body facing surface 19 of chassis 11) and the bodyside liner 28 is disposed at the body facing surface 19 of the chassis 11. In an embodiment, the bodyside liner 28 can be bonded to the outer cover 26 in a superposed relation by any suitable means such as, but not limited to, adhesives, ultrasonic bonds, thermal bonds, pressure bonds, or other conventional techniques. The outer cover 26 can define a length in a longitudinal direction 30, and a width in the lateral direction 32, which, in the illustrated embodiment, can coincide with the length and width of the absorbent article 10. As illustrated in FIGS. 2 and 2A, the absorbent article 10, 110 can have a longitudinal axis 29 extending in the longitudinal direction 30, and a lateral axis 31 extending in the lateral direction 32. The lateral axis 31 is located midway between the front waist edge 22 and the rear waist edge 24, separating article 10 into front half section 25 and rear half section 27. As shown in FIG. 3, the absorbent article 10 also has a vertical or z-direction axis 100 extending in the vertical direction 33.

The chassis 11 can include an absorbent body 34. The absorbent body 34 can be disposed between the outer cover 26 and the bodyside liner 28. The absorbent body 34 can have longitudinal edges, 36 and 38, which, in an embodiment, can form portions of the longitudinal side edges, 18 and 20, respectively, of the absorbent article 10, 110. The absorbent body 34 can have a first end edge 40 that is opposite a second end edge 42, respectively, which, in an embodiment, can form portions of the waist edges, 22 and 24, respectively, of the absorbent article 10. In some embodiments, the first end edge 40 can be in the front waist region 12. In some embodiments, the second end edge 42 can be in the rear waist region 14. In an embodiment, the absorbent body 34 can have a length and width that are the same as or less than the length and width of the absorbent article 10, 110. The bodyside liner 28, the outer cover 26, and the absorbent body 34 can form part of an absorbent assembly 44. In the absorbent article 110 of FIGS. 1A and 2A, the absorbent panel 17 can form the absorbent assembly 44. The absorbent assembly 44 can also include a fluid transfer layer 46 and a fluid acquisition layer (not shown) between the bodyside liner 28 and the fluid transfer layer 46 as is known in the art. The absorbent assembly 44 can also include a spacer layer 48 (as shown in FIG. 3) disposed between the absorbent body 34 and the outer cover 26.

The absorbent article 10, 110 can be configured to contain and/or absorb liquid, solid, and semi-solid body exudates discharged from the wearer. To this end, the absorbent article 10, 110 is fitted with a pair of containment flaps 50, 52 which are configured to provide a barrier to the lateral flow of body exudates. In some further embodiments, the absorbent article 10, 110 can optionally include a waistband, such as waistband 202 depicted in dashed lines in FIGS. 2-5 which cooperates with the containment flaps 50, 52 to form a pocket to further retain body exudates. See, for example, PCT/US15/23620, Attorney Docket Number 64967740PCT01 filed 31 Mar. 2015; PCT/US15/23596, Attorney Docket Number 64965789PCT01 filed 31 Mar. 2015; PCT/US15/23637, Attorney Docket Number 64967082PCT01 filed 31 Mar. 2015; PCT/US15/38271, Attorney Docket Number 64969698PCT01 filed 29 Jun. 2015; PCT/US15/47672, Attorney Docket Number 64970360PCT01 filed 31 Aug. 2015; and 62/212,051, Attorney Docket Number 64968524US01 filed 31 Aug. 2015 each of which is incorporated herein by reference in its entirety. In some embodiments, the waist containment member can be disposed in the rear waist region 14 of the absorbent article 10, 110.

The absorbent article 10, 110 can further include leg elastic members 60, 62 as are known to those skilled in the art. The leg elastic members 60, 62 can be attached to the outer cover 26 and/or the bodyside liner 28 along the opposite longitudinal side edges, 18 and 20, and positioned in the crotch region 16 of the absorbent article 10, 110. The leg elastic members 60, 62 can be parallel to the longitudinal axis 29 as shown in FIGS. 2 and 2A or can be curved as is known in the art. The leg elastic members 60, 62 can provide elasticized leg cuffs.

Additional details regarding each of these elements of the absorbent article 10, 110 described herein can be found below and with reference to the FIGS. 1-7.

Outer Cover:

The outer cover 26 and/or portions thereof can be breathable and/or liquid impermeable. The outer cover 26 and/or portions thereof can be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the outer cover 26 can be constructed of a microporous polymeric film, such as polyethylene or polypropylene.

In an embodiment, the outer cover 26 can be a single layer of a liquid impermeable material, such as a polymeric film. In an embodiment, the outer cover 26 can be suitably stretchable, and more suitably elastic, in at least the lateral direction 32 of the absorbent article 10, 110. In an embodiment, the outer cover 26 can be stretchable, and more suitably elastic, in both the lateral 32 and the longitudinal 30 directions. In an embodiment, the outer cover 26 can be a multi-layered laminate in which at least one of the layers is liquid impermeable. In some embodiments, the outer cover 26 can be a two layer construction, including an outer layer (not shown) and an inner layer (not shown) which can be bonded together such as by a laminate adhesive. Suitable laminate adhesives can be applied continuously or intermittently as beads, a spray, parallel swirls, or the like, but it is to be understood that the inner layer can be bonded to the outer layer by other bonding methods, including, but not limited to, ultrasonic bonds, thermal bonds, pressure bonds, or the like.

The outer layer of the outer cover 26 can be any suitable material and may be one that provides a generally cloth-like texture or appearance to the wearer. An example of such material can be a 100% polypropylene bonded-carded web with a diamond bond pattern available from Sandler A. G., Germany, such as 30 gsm Sawabond 4185® or equivalent. Another example of material suitable for use as an outer layer of an outer cover 26 can be a 20 gsm spunbond polypropylene non-woven web. The outer layer may also be constructed of the same materials from which the bodyside liner 28 can be constructed as described herein and it also may be apertured to enhance evaporation of urine in the event the inner layer is vapor permeable.

The liquid impermeable inner layer of the outer cover 26 (or the liquid impermeable outer cover 26 where the outer cover 26 is of a single-layer construction) can be either vapor permeable (i.e., “breathable”) or vapor impermeable. The liquid impermeable inner layer (or the liquid impermeable outer cover 26 where the outer cover 26 is of a single-layer construction) can be manufactured from a thin plastic film. The liquid impermeable inner layer (or the liquid impermeable outer cover 26 where the outer cover 26 is of a single-layer construction) can inhibit liquid body exudates from leaking out of the absorbent article 10, 110 and wetting articles, such as bed sheets and clothing, as well as the wearer and caregiver.

In some embodiments, where the outer cover 26 is of a single layer construction, it can be embossed and/or matte finished to provide a more cloth-like texture or appearance. The outer cover 26 can permit vapors to escape from the absorbent article 10, 110 while preventing liquids from passing through. A suitable liquid impermeable, vapor permeable material can be composed of a microporous polymer film or a non-woven material which has been coated or otherwise treated to impart a desired level of liquid impermeability.

Absorbent Body:

The absorbent body 34 can be suitably constructed to be generally compressible, conformable, pliable, non-irritating to the wearer's skin and capable of absorbing and retaining liquid body exudates. The absorbent body 34 can be manufactured in a wide variety of sizes and shapes (for example, rectangular, trapezoidal, T-shape, I-shape, hourglass shape, etc.) and from a wide variety of materials. The size and the absorbent capacity of the absorbent body 34 should be compatible with the size of the intended wearer (infants to adults) and the liquid loading imparted by the intended use of the absorbent article 10, 110. The absorbent body 34 can have a length and width that can be less than or equal to the length and width of the absorbent article 10, 110.

In an embodiment, the absorbent body 34 can be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an embodiment, the absorbent body 34 can be a matrix of cellulosic fluff and superabsorbent material. In an embodiment, the absorbent body 34 may be constructed of a single layer of materials, or in the alternative, may be constructed of two or more layers of materials.

Various types of wettable, hydrophilic fibers can be used in the absorbent body 34. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. Suitable superabsorbent materials can be selected from natural, synthetic, and modified natural polymers and materials. The superabsorbent materials can be inorganic materials, such as silica gels, or organic compounds, such as cross-linked polymers. In an embodiment, the absorbent body 34 can be free of superabsorbent material or in an alternate embodiment be comprised entirely of superabsorbent material.

If a spacer layer 48 is present, the absorbent body 34 can be disposed on the spacer layer 48 and superposed over the outer cover 26. The spacer layer 48 can be bonded to the outer cover 26, for example, by adhesive. In some embodiments, a spacer layer 48 may not be present and the absorbent body 34 can directly contact the outer cover 26 and can be directly bonded to the outer cover 26. However, it is to be understood that the absorbent body 34 may be in contact with, and not bonded with, the outer cover 26 and remain within the scope of this disclosure. In an embodiment, the outer cover 26 can be composed of a single layer and the absorbent body 34 can be in contact with the singer layer of the outer cover 26. In some embodiments, at least a portion of a layer, such as but not limited to, a fluid transfer layer 46 and/or a spacer layer 48, can be positioned between the absorbent body 34 and the outer cover 26, such as illustrated in FIG. 3. The absorbent body 34 can be bonded to the fluid transfer layer 46 and/or the spacer layer 48. Typically the absorbent body 34 will be completely enveloped by a core wrap material such as a tissue wrap or a nonwoven material such a meltblown web, a spunbond web or both.

Bodyside Liner:

The bodyside liner 28 of the absorbent article 10, 110 can overlay the absorbent body 34 and the outer cover 26 and can isolate the wearer's skin from liquid waste retained by the absorbent body 34. In various embodiments, a fluid transfer layer 46 can be positioned between the bodyside liner 28 and the absorbent body 34. In various embodiments, an acquisition layer (not shown) can be positioned between the bodyside liner 28 and the absorbent body 34 or a fluid transfer layer 46, if present. In various embodiments, the bodyside liner 28 can be bonded to the acquisition layer, or to the fluid transfer layer 46 if no acquisition layer is present, via adhesive and/or by a point fusion bonding. The point fusion bonding may be selected from ultrasonic, thermal, pressure bonding, and combinations thereof.

In an embodiment, the bodyside liner 28 can extend beyond the absorbent body 34 and/or a fluid transfer layer 46, if present, and/or an acquisition layer, if present, and/or a spacer layer 48, if present, to overlay a portion of the outer cover 26 and can be bonded thereto by any method deemed suitable, such as, for example, by being bonded thereto by adhesive, to substantially enclose the absorbent body 34 between the outer cover 26 and the bodyside liner 28. It is contemplated that the bodyside liner 28 may be narrower than the outer cover 26. However, in other embodiments, the bodyside liner 28 and the outer cover 26 may be of the same dimensions in width and length, for example, as depicted in the embodiments illustrated in FIG. 1. In other embodiments, the bodyside liner 28 can be of greater width than the outer cover 26. It is also contemplated that the bodyside liner 28 may not extend beyond the absorbent body 34 and/or may not be secured to the outer cover 26. In some embodiments, the bodyside liner 28 can wrap at least a portion of the absorbent body 34, including wrapping around both longitudinal edges 36, 38 of the absorbent body 34, and/or one or more of the end edges 40, 42. It is further contemplated that the bodyside liner 28 may be composed of more than one segment of material such as a central region of material which is different from one or both of the lateral regions of the bodyside liner 28 (not shown). The bodyside liner 28 can be of different shapes, including rectangular, hourglass, or any other shape. The bodyside liner 28 can be suitably compliant, soft feeling, and non-irritating to the wearer's skin and can be the same as or less hydrophilic than the absorbent body 34 to permit body exudates to readily penetrate through to the absorbent body 34 and provide a relatively dry surface to the wearer.

The bodyside liner 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the bodyside liner 28. The bodyside liner 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, can include more than one layer of fabrics, films, and/or webs, as well as combinations thereof. For example, the bodyside liner 28 can include a support layer and a projection layer that can be hydroentagled. The projection layer can include hollow projections, such as those disclosed in U.S. Pat. No. 9,327,473 to Kirby et al.

For example, the bodyside liner 28 can be composed of a meltblown or spunbond web of polyolefin fibers. Alternatively, the bodyside liner 28 can be a bonded-carded web composed of natural and/or synthetic fibers. The bodyside liner 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire bodyside liner 28 or it can be selectively applied to particular sections of the bodyside liner 28.

In an embodiment, a bodyside liner 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a bodyside liner 28 can be a spunbond substrate with a basis weight from about 10 or 12 to about 15 or 20 gsm. In an embodiment, a bodyside liner 28 can be a 12 gsm spunbond-meltblown-spunbond substrate having 10% meltblown content applied between the two spunbond layers.

Although the outer cover 26 and bodyside liner 28 can include elastomeric materials, it is contemplated that the outer cover 26 and the bodyside liner 28 can be composed of materials which are generally non-elastomeric. In an embodiment, the bodyside liner 28 can be stretchable, and more suitably elastic. In an embodiment, the bodyside liner 28 can be suitably stretchable and more suitably elastic in at least the lateral or circumferential direction of the absorbent article 10, 110. In other aspects, the bodyside liner 28 can be stretchable, and more suitably elastic, in both the lateral and the longitudinal directions 32, 30, respectively.

Containment Flaps:

In an embodiment, the absorbent article 10, 110 can include a pair of containment flaps 50, 52. Examples of containment flaps can be found in U.S. Pat. No. 9,259,362 granted 16 Feb. 2016 Robert L. Popp et al. and U.S. Pat. No. 9,168,181 granted 27 Oct. 2015 Robert L. Popp et al. each of which is incorporated herein by reference in its entirety.

The containment flaps 50, 52 can be formed separately from the absorbent chassis 11 and attached to the chassis 11 or can be formed integral to the chassis 11. In some embodiments, the containment flaps 50, 52 can be secured to the chassis 11 of the absorbent article 10, 110 in a generally parallel, spaced relation with each other laterally inward of the leg openings to provide a barrier against the flow of body exudates. One containment flap 50 can be on a first side of the longitudinal axis 29 and the other containment flap 52 can be on a second side of the longitudinal axis 29. In an embodiment, the containment flaps 50, 52 can extend generally in a longitudinal direction 30 from the front waist region 12 of the absorbent article 10, through the crotch region 16 to the rear waist region 14 of the absorbent article 10. In some embodiments, the containment flaps 50, 52 can extend in a direction substantially parallel to the longitudinal axis 29 of the absorbent article 10, however, in other embodiments, the containment flaps 50, 52 can be curved, as is known in the art. In other embodiments, such as the absorbent article 110 in FIGS. 1A and 2A, the containment flaps 50, 52 can be disposed on the absorbent panel 17 in the crotch region 16.

In embodiments where the containment flaps 50, 52 are coupled to the chassis 11, the containment flaps 50, 52 can be bonded to the bodyside liner 28 with a barrier adhesive 49, as shown in FIG. 3 or the containment flaps 50, 52 can be bonded to the outer cover 26 with a barrier adhesive 49 in some embodiments where the bodyside liner 28 does not extend the full lateral width of the outer cover 26. Of course, the containment flaps 50, 52 can be bonded to other components of the chassis 11 and can be bonded with other suitable means other than a barrier adhesive 49 or with several areas or beads of barrier adhesive. In addition to adhesive, other bonding means can be employed including, but not limited to, pressure bonding and ultrasonic bonding. The containment flaps 50, 52 can be constructed of a fibrous material which can be similar to the material forming the bodyside liner 28, including, but not limited to a spunbond-meltblown-spunbond (“SMS”) material. Other conventional materials, including, but not limited to, polymer films, can also be employed. In addition, laminates of materials including multiple layers of film and/or nonwovens can be used to form the containment flap material.

The containment flaps 50, 52 in an embodiment such as is shown in FIGS. 1-5 can each include a base portion 64 and a projection portion 66. The base portion 64 and the projection portion 66 can be formed from the same materials or from different materials. In an embodiment, the containment flaps 50, 52 can comprise only the projection portion 66 with the projection portion 66 defining the entire containment flap 50, 52. In such situations, the base portion 64 may be eliminated or it may be formed from one of the other components such as an extension of the bodyside liner 28, the outer cover 26 or from a separate and distinct piece of material (not shown).

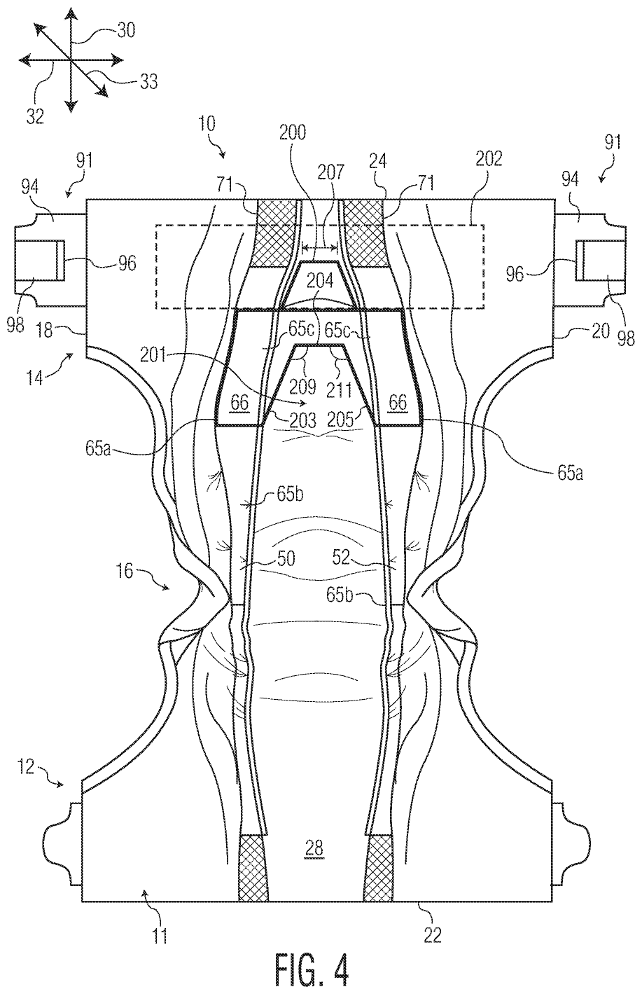

The base portion 64 can be bonded to the chassis 11, for example, to the bodyside liner 28 or the outer cover 26 or another component of the chassis 11. The base portion 64 can include an interior end 64a and an exterior end 64b. The projection portion 66 can be separated from the base portion 64 at the interior end 64a of the base portion 64. As used in this context, the projection portion 66 is separated from the base portion 64 at the interior end 64a of the base portion 64 in that the interior end 64a of the base portion 64 defines a transition between the projection portion 66 and the base portion 64. The interior end 64a of the base portion 64 can be located near and/or utilize all or a portion of the barrier adhesive 49 or a separate barrier adhesive 49 or other attachment means. In some embodiments, the exterior ends 64b of the base portion 64 of the containment flaps 50, 52 can laterally extend to the respective longitudinal side edges 18, 20 of the absorbent article 10. In other embodiments, the exterior ends 64b of the base portion 64 can end laterally inward of the respective longitudinal side edges 18, 20 of the absorbent article 10, 110. The containment flaps 50, 52 also each include a projection portion 66 that is configured to extend away from the body facing surface 19 of the chassis 11 at least in the crotch region 16 when the absorbent article 10 is in a relaxed configuration, as illustrated in FIGS. 3 and 4. The containment flaps 50, 52 can include a tack-down region 71 in either or both of the front waist region 12 and the rear waist region 14 where the projection portion 66 is coupled to the body facing surface 19 of the chassis 11, as can be seen in FIG. 2.

In a more simplified version, the base portion 64 of the containment flaps 50, 52 can be eliminated (not shown) or can be made from a separate piece of material such that the projection portion 66 forms the entire containment flap 50, 52. In this case, the containment flaps 50, 52 can be regarded as having a proximal edge 65a and a distal edge 65b joined by a medial section 65c. See in particular, FIGS. 2, 3, and 4. The proximal edge 65a can be attached to the chassis 11 including direct or indirect attachment to any of the components including the bodyside liner 28, the outer cover 26 or any of the other components of the absorbent article 10. Thus, the barrier adhesive 49 can be used to attach the proximal edge 65a to the chassis 11 and this barrier adhesive 49 can be the same as or separate from the barrier adhesive used to attach the interior end 64a of the base portion 64 to the chassis 11.

It is contemplated that the containment flaps 50, 52 can be of various configurations and shapes, and can be constructed by various methods. For example, the containment flaps 50, 52 of FIG. 2 depict a vertical containment flap 50, 52 with a tack-down region 71 in both the front and rear waist regions 12, 14 where the projection portion 66 of each containment flap 50, 52 is tacked down to the bodyside liner 28 towards or away from the longitudinal axis 29 of the absorbent article 10. However, the containment flaps 50, 52 can include a tack-down region 71 where the projection portion 66 of each of the containment flaps 50, 52 is folded back upon itself and coupled to itself and the bodyside liner 28 in a “C-shape” configuration, as described in U.S. Pat. No. 5,895,382 to Robert L. Popp et al. As yet another alternative, it is contemplated that the containment flaps 50, 52 can be constructed in a “T-shape” configuration, such as described in U.S. Pat. No. 9,259,362 by Robert L. Popp et al. Such a configuration can also include a tack-down region 71 in either or both of the front and rear waist regions 12, 14, respectively. Of course, other configurations of containment flaps 50, 52 can be used in the absorbent article 10, 110 and still remain within the scope of this disclosure.

The containment flaps 50, 52 can include one or more flap elastic members 68, such as the two flap elastic strands depicted in FIGS. 2 and 3. Of course, while two elastic members 68 are shown in each containment flap 50, 52, it is contemplated that the containment flaps 50, 52 can be configured with one or three or more elastic members 68. As shown in the Figures, the elastic members 68 are located adjacent the flap distal edges 65b but they can also be located in any other portion of the containment flaps including adjacent the flap proximal portion 65a and the flap medial portion 65c.

Suitable elastic materials for the flap elastic members 68 can include, but are not limited to, spandex elastomeric strands, sheets, strands, or ribbons of natural or synthetic rubber, thermoplastic elastomeric materials, or heat activated elastomeric materials. The elastic members 68 can be any elastomeric material capable of being elongated at least about 50 percent, desirably about 350 percent, and capable of recovering to within at least about 250 percent, and desirably about 150 percent of its original length after being elongated about 300 percent. The elastic members 68 can be a spandex elastomeric strand(s) such as, for example, a LYCRA thread commercially available from E. I. DuPont de Nemours and Co. Alternatively, the elastic members 68 can be composed of a thermoplastic elastomer or a natural or a synthetic rubber commercially available from J.P.S. Elastomerics Corp. Alternatively, the elastic members 68 can also be composed of a heat activated elastic material such as PEBAX, commercially available from Atochem, Inc., which can be activated with heat treatment after the elastic members 68d are secured to the containment flaps 50, 52.

Alternatively or additionally, the containment flaps 50, 52 can be composed of a material exhibiting elastic properties itself thereby eliminating the need for the use of separate flap elastic members 68. In an alternate embodiment, the containment flaps 50, 52, and in particular the projection portions 66, can be formed from an elastic material or laminate such as a stretch film laminate as described herein which optionally can be supplemented with flap elastics adjacent the distal edges 65b, alternately adjacent the proximal edges 65a, alternately adjacent the medial section 65c/41 or a combination of any or all of the foregoing regions of the projection portions 66 forming the containment flaps 50, 52. Example suitable materials that may be used as containment flaps 50, 52 include vertical film laminate materials, stretch film laminate materials, or elastic laminate structures. One specific example material is the elastic film laminate material called SABBEEL herein, which is described in U.S. Pat. No. 8,287,677, titled “Printable Elastic Composite” and is expressly incorporated herein in its entirety. Additionally, it should be understood that these are only example materials. More generally, any material having at least some of the below described properties are contemplated by this disclosure for use as containment flaps 50, 52.

The flap elastic members 68, as illustrated in FIGS. 2 and 3, can have two strands of elastomeric material extending longitudinally in the projection portion 66 of the containment flaps 50, 52, in generally parallel, spaced relation with each other. The elastic members 68 can be within the containment flaps 50, 52 while in an elastically contractible condition such that contraction of the strands gathers and shortens the projection portions 66 of the containment flaps 50, 52 in the longitudinal direction 30. As a result, the elastic members 68 can bias the projection portions 66 of the containment flaps 50, 52 to extend away from the body facing surface 45 of the absorbent assembly 44 in a generally upright orientation of the containment flaps 50, 52, especially in the crotch region 16 of the absorbent article 10, when the absorbent article 10 is in a relaxed configuration.

During manufacture of the containment flaps 50, 52 at least a portion of the elastic members 68 can be bonded to the containment flaps 50, 52 while the elastic members 68 are elongated. The percent elongation of the elastic members 68 can be, for example, about 110% to about 350%. The elastic members 68 can be coated with adhesive while elongated to a specified length prior to attaching the elastic members 68 to the containment flaps 50, 52. In a stretched condition, the length of the elastic members 68 which have adhesive coupled thereto can provide an active flap elastic region 70 in the containment flaps 50, 52, as labeled in FIG. 2, which will gather upon relaxation of the absorbent article 10. The active flap elastic region 70 of containment flaps 50, 52 can be of a longitudinal length that is less than the length of the absorbent article 10, 110. In this exemplary method of bonding the elastic members 68 to the containment flaps 50, 52, the portion of the elastic members 68 not coated with adhesive will retract after the elastic members 68 and the absorbent article 10 are cut in manufacturing to form an individual absorbent article 10. As noted above, the relaxing of the elastic members 68 in the active flap elastic region 70 when the absorbent article 10, 110 is in a relaxed condition can cause each containment flap 50, 52 to gather and cause the projection portion 66 of each containment flap 50, 52 to extend away from the body facing surface 19 of the chassis 11 (e.g., the body facing surface 45 of the absorbent assembly 44 or the body facing surface 56 of the bodyside liner 28), as depicted in FIGS. 2 and 3.

Of course, the elastic members 68 can be bonded to the containment flaps 50, 52 in various other ways as known by those of skill in the art to provide an active flap elastic region 70, which is within the scope of this disclosure. Additionally, the active flap elastic regions 70 can be shorter or longer than depicted herein, including extending to the front waist edge 22 and the rear waist edge 24, and still be within the scope of this disclosure.

Leg Elastics:

Leg elastic members 60, 62 can be secured to the outer cover 26, such as by being bonded thereto by laminate adhesive, generally laterally inward of the longitudinal side edges, 18 and 20, of the absorbent article 10, 110. The leg elastic members 60, 62 can form elasticized leg cuffs that further help to contain body exudates. In an embodiment, the leg elastic members 60, 62 may be disposed between inner and outer layers (not shown) of the outer cover 26 or between other layers of the absorbent article 10, for example, between the base portion 64 of each containment flap 50, 52 and the bodyside liner 28 as depicted in FIGS. 2, 2A and 3, between the base portion 64 of each containment flap 50, 52 and the outer cover 26, or between the bodyside liner 28 and the outer cover 26. The leg elastic members 60, 62 can be one or more elastic components near each longitudinal side edge 18, 20. For example, the leg elastic members 60, 62 as illustrated herein each include two elastic strands. A wide variety of elastic materials may be used for the leg elastic members 60, 62.

Suitable elastic materials can include sheets, strands or ribbons of natural rubber, synthetic rubber, or thermoplastic elastomeric materials. The elastic materials can be stretched and secured to a substrate, secured to a gathered substrate, or secured to a substrate and then elasticized or shrunk, for example, with the application of heat, such that the elastic retractive forces are imparted to the substrate. Additionally, it is contemplated that the leg elastic members 60, 62 can be formed with the containment flaps 50, 52, and then attached to the chassis 11 in some embodiments. Of course, the leg elastic members 60, 62 can be omitted from the absorbent article 10, 110 without departing from the scope of this disclosure.

Fastening System:

In an embodiment, the absorbent article 10, can include a fastening system. The fastening system can include one or more back fasteners 91 and one or more front fasteners 92. The embodiments being shown in FIGS. 1 and 2 depict embodiments with one front fastener 92. Portions of the fastening system may be included in the front waist region 12, rear waist region 14, or both.

The fastening system can be configured to secure the absorbent article 10 about the waist of the wearer in a fastened condition as shown in FIG. 1 and help maintain the absorbent article 10 in place during use. In an embodiment, the back fasteners 91 can include one or more materials bonded together to form a composite ear as is known in the art. For example, the composite fastener may be composed of a stretch component 94, a nonwoven carrier or hook base 96, and a fastening component 98, as labeled in FIG. 2.

According to aspects of the present disclosure, some embodiments of absorbent articles 10, 110 may include connecting portion 200, which connects containment flaps 50, 52 in the rear half section 27 of the articles 10, 110 as seen in FIGS. 2 and 2A. The connecting portion 200 cooperates with containment flaps 50, 52 to form opening 201 to absorbent assembly 44. Opening 201 is designed to allow exudates from a BM pass by BM flaps 50, 52 to absorbent assembly 44, where the exudates are ideally absorbed, while still providing a large area of coverage of the wearer's skin forming a barrier between the skin and the BM as exudates spread on absorbent assembly 44.

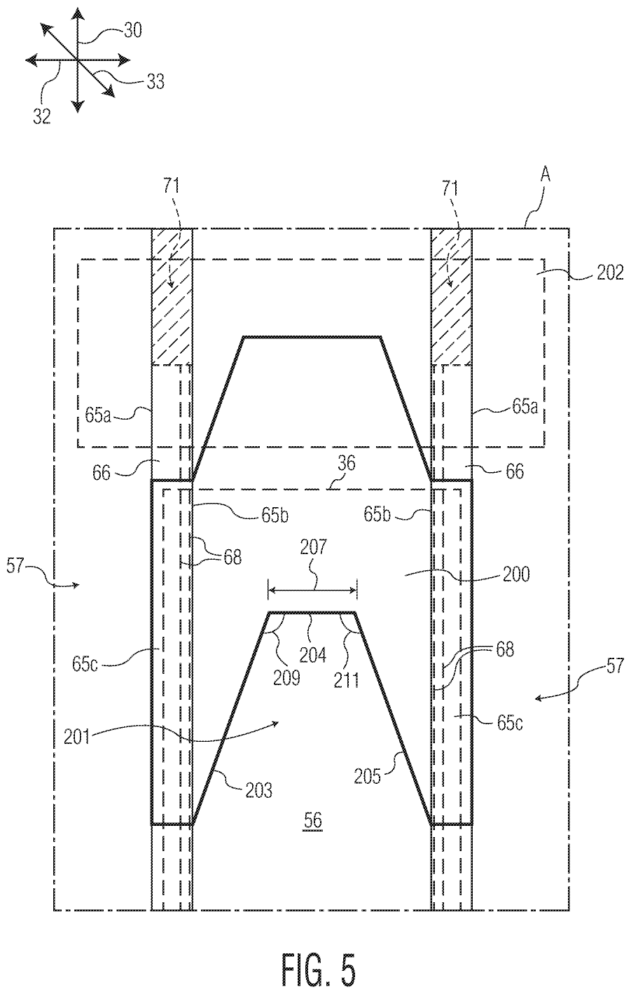

FIG. 5 depicts a close-up of region A in the rear half section 27 of article 10 of FIG. 2 to provide greater detail on connecting portion 200 and opening 201. As seen in FIGS. 2-5, opening 201 can be defined by laterally opposed edges 203 and 205. Laterally opposed edges 203 and 205 may generally extend in the longitudinal direction 30, although in some embodiments laterally opposed edges 203 and 205 may form an angle with respect to longitudinal axis, as seen in FIG. 5. Laterally opposed edges 203 and 205 may further have a minimum separation distance 207 within the rear half section 27. Minimum separation distance 207 may be the smallest distance that laterally opposed edges 203 and 205 are separated in the lateral direction 32 in the rear half section 27. In different embodiments, minimum separation distance 207 may range between 0 mm and up to the distance between projection portions 66 of containment flaps 50, 52 when the article is in a laid flat configuration. In further embodiments, minimum separation distance 207 may range between about one-quarter and about three-quarters of the distance between the projection portions 66 of the containment flaps 50, 52, or the distance between the distal flap edges 65b of the projection portions 66 of the containment flaps 50, 52, when the article is in a laid flat configuration. In further embodiments, minimum separation distance 207 may range between about one-third and about two-thirds of the distance between the projection portions 66 of the containment flaps 50, 52, or the distance between the distal flap edges 65b of the projection portions 66 of the containment flaps 50, 52, when the article is in a laid flat configuration. In still additional embodiments, minimum separation distance 207 may range between about 0 mm and about 250 mm. As will be described below with respect to FIGS. 8-12, the minimum separation distance 207 may be an important feature affecting how articles of the present disclosure, such as articles 10 and 110, fit on a wearer and ultimately how the articles 10 and 110 perform to allow BM to pass through opening 201 yet still protect the skin of the wearer.

In some embodiments, opening 201 may further be defined by laterally extending back edge 204. Laterally extending back edge 204 may define the rear-most part of opening 201 and may extend generally in the lateral direction 32, but does not need to be parallel with lateral axis 31. For instance, in some embodiments, laterally extending back edge 204 may form an angle with lateral axis 31. Additionally, laterally opposed edges 203 and 205 may form angles 209, 211 with laterally extending back edge 204 where laterally opposed edges 203 and 205 connect to laterally extending back edge 204. The specific values for angles 209, 211 may result from the construction of connecting portion 200, in terms of minimum separation distance 207 and the lengths of laterally opposed edges 203 and 205. However, in at least some embodiments, angles 209, 211 may range between about 60 degrees and about 85 degrees. In further embodiments, angles 209, 211 may range between about 65 degrees and about 75 degrees. However, it should be understood that angles 209, 211 are not limited to these specified values. Rather, angles 209, 211 may be any value achieved as a result for suitable chosen values of minimum separation distance 207 and the lengths of laterally opposed edges 203 and 205. Additionally, although in some embodiments angles 209, 211 may be the same, this is not necessary in all embodiments. For instance, in some embodiments, angles 209, 211 may differ from each other, although may generally differ by less than about 25 degrees. As one illustrative example, angle 209 may be 75 degrees, while angle 211 may be 60 degrees.

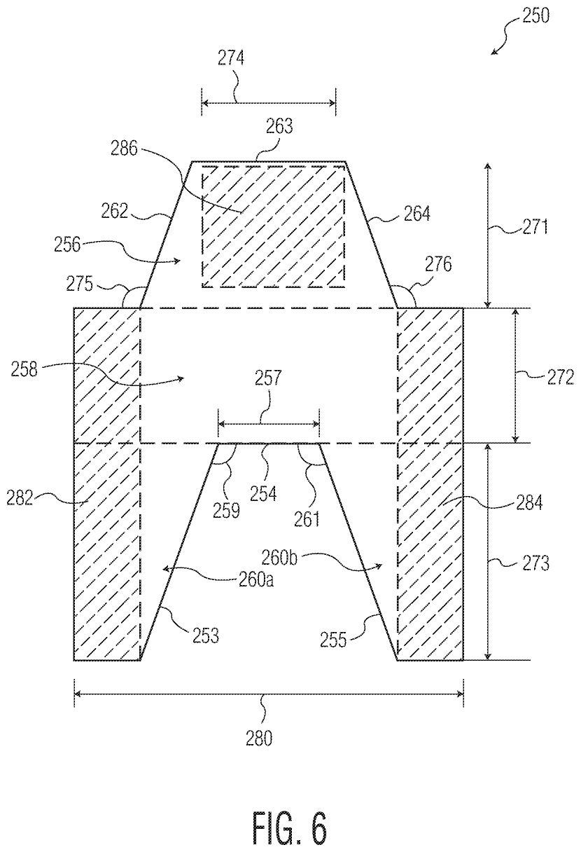

In general, connecting portion 200 may be formed through any desired means. In some embodiments, connecting portion 200 may be integral with containment flaps 50, 52. For instance, containment flaps 50, 52 may originally comprise a continuous web of material with a section that spans between elastic members 68 of each containment flap 50, 52. In such embodiments, a portion of the intermediate web between elastic members 68 of each containment flap 50, 52 may be removed to form opening 201. In other embodiments, however, connecting portion 200 may be a discrete patch of material that is attached across containment flaps 50, 52, as described below with respect to FIG. 6.

FIG. 6 depicts an exemplary discrete patch 250 that may be used according to aspects of the present disclosure to form articles 10, 110 including connecting portion 200 and opening 201. Discrete patch 250 may comprise tongue portion 256, base portion 258, and leg portions 260a, 260b. However, it should be understood that each of tongue portion 256, base portion 258, and leg portions 260a, 260b need not be separate components that are bonded together. Rather, discrete patch 250 may be a single piece of continuous material, with tongue portion 256, base portion 258, and leg portions 260a, 260b called out for ease of description.

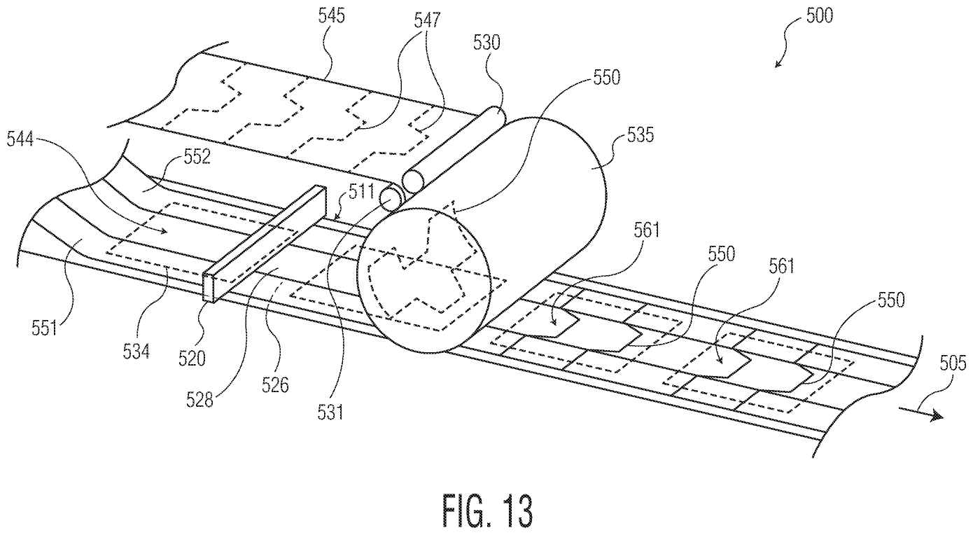

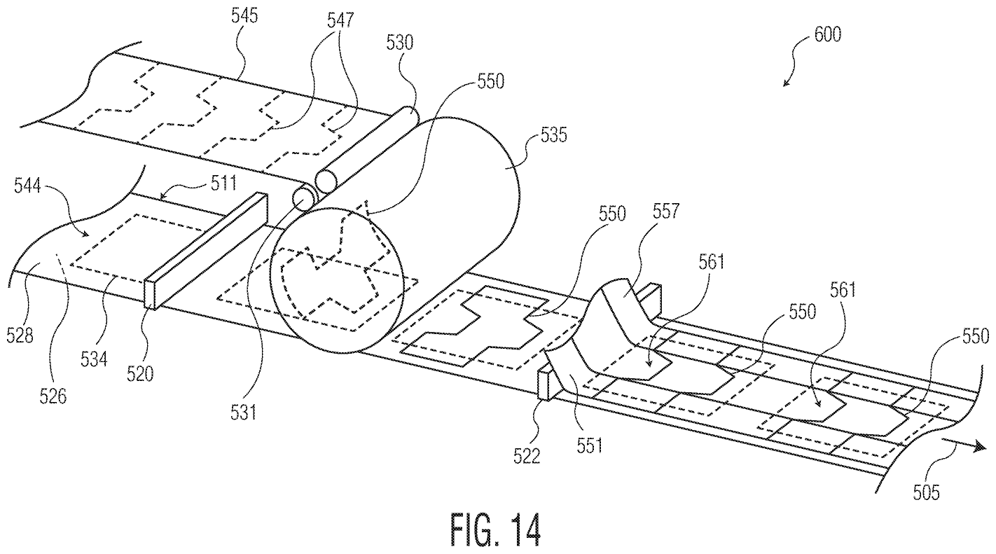

In general, tongue portion 256 may extend away base portion 258 in a first direction while leg portions 260a, 260b extend away from base portion 258 in a second direction generally opposite the first direction. Tongue portion 256 may comprise side edges 262, 264 and top edge 263 and may extend away from base portion a distance 271 any suitable amount for a given absorbent article. Because patch 250 may be used on many varieties of absorbent articles of different sizes, distance 271 may span a relatively large range of values. However, in at least some embodiments, distance 271 may be between about 15 mm and about 100 mm. Top edge 263 may additionally have a lateral extent 274. Again, as patch 250 may be used on many varieties of absorbent articles of different sizes, lateral extent 274 may span a relatively large range of values. According to some embodiments of the present disclosure, lateral extent 274 may equal minimum separation distance 257. However, this is not necessary in all embodiments. In these embodiments, patch 250 may be formed using a no-waste process, as is described below with respect to FIGS. 13 and 14. Further, in at least some embodiments, lateral extent 274 may be the same as a lateral extent of tongue portion 256 where tongue portion 256 connects to base portion 258. That is, tongue portion 256 may have a square shape instead of the trapezoid shape depicted in FIG. 6. In still further embodiments, lateral extent 274 may be greater than a lateral extent of tongue portion 256 where tongue portion 256 connects to base portion 258. In such embodiments, tongue portion 256 may look like an upside-down trapezoid.

As seen in FIG. 6, in at least some embodiments, side edges 262, 264 may extend away from base portion 258 at an angle, although this is not necessary in all embodiments. In other embodiments, side edges 262, 264 may be perpendicular with base portion 258. However, in embodiments where side edges 262, 264 extend away from base portion 258 at an angle, side edges 262, 264 may form angles 275, 276 with base portion 258. In different embodiments, angles 275, 276 may range between about 90 degrees and about 165 degrees. In other embodiments, angles 275, 276 may range between about 115 degrees and about 145 degrees. In embodiments where patch 250 is formed in a nested, no waste manner, angles 275, 276 may be the same as angles 259, 261, and angles 259, 261 may be a result of the chosen minimum separation distance 257 and lengths of angled internal edges 253 and 255.

Base portion 258 may generally connect tongue portion 256 with leg portions 260a, 260b. In the example of FIG. 6, base portion 258 is depicted as rectangular. However, in other embodiments, base portion 258 may have be another shape, for instance a trapezoid. Additionally, in at least some embodiments, base portion 258 and tongue portion 256 may be configured into a combined section where there is no distinction between where base portion 258 ends and tongue portion 256 begins. Base portion 258 may further have a longitudinal dimension 272 which may span a relatively large range of values. However, in at least some embodiments, longitudinal dimension 272 may range between about 5 mm and about 100 mm in various embodiments. In still further embodiments, longitudinal dimension 272 may range between about 15 mm and about 60 mm.

As mentioned above, leg portions 260a, 260b may extend away from base portion 258 and may each have a longitudinal dimension 273. In general, longitudinal dimension 273 may have any value suitable for a given absorbent article, which may vary relatively greatly between different sized absorbent articles. In at least some embodiments, longitudinal dimension 273 may equal to between about 30% and about 75% of the distance between a central lateral fold line of an absorbent article to which patch 250 is to be attached and a back waist edge of the absorbent article. In further embodiments, longitudinal dimension 273 may be equal to between about 45% and about 65% of the distance between a central lateral fold line of an absorbent article to which patch 250 is to be attached and a back waist edge of the absorbent article. As will be described in more detail below, in some embodiments, patch 250 may be applied to an absorbent article in a stretched state. In such embodiments, longitudinal dimension 273 have a value equal to between about 40% to about 80% of the distance between a central lateral fold line of the absorbent article to which patch 250 is attached and a back waist edge of the absorbent article, when the absorbent article is in a stretched and laid-flat configuration. In further embodiments, longitudinal dimension 273 have a value equal to between about 55% to about 75% of the distance between a central lateral fold line of the absorbent article to which patch 250 is attached and a back waist edge of the absorbent article, when the absorbent article is in a stretched and laid-flat configuration.

Additionally, in some embodiments, each leg portion 260a, 260b may have at least one angled edge, such as for example angled internal edges 253 and 255. In general, angled internal edges 253 and 255 may extend in the longitudinal direction 30, but may not necessarily be perpendicular to longitudinal axis 29. Accordingly, angled internal edges 253 and 255 may form angles 259, 261 where leg portions 260a, 260b connect with base portion 258. Additionally, where leg portions 260a, 260b connect with base portion 258, leg portions 260a, 260b may be separated by a separation distance 257, which is the length of bottom portion 254 of base portion 258.

As can be seen, in embodiments where connecting portion 200 is a discrete patch of material, such as patch 250, opening 251 may be defined by angled internal edges 253 and 255 and bottom portion 254 of base portion 258. Accordingly, in various embodiments according to the present disclosure, angles 259, 261 may range between the same values as angles 209, 211 described previously with respect to FIGS. 2-4, and separation distance 257 may range between the same values as minimum separation distance 207.

Patch 250 further has an overall lateral dimension 280. This lateral dimension 280 may generally be enough to allow patch 250 to span between containment flaps 50, 52 of absorbent articles of the present disclosure. For instance, patch 250 may be useful in absorbent articles of differing forms and sizes, such as different sized diapers or adult absorbent articles. Accordingly, lateral dimension 280 may comprise any suitable value and may be chosen such that patch 250 spans the distance between containment flaps on the desired absorbent article. In at least some embodiments, lateral dimension 280 may be chosen such that leg portions 260a, 260b each encompass at least the projection portions 66 of a respective containment flap on the absorbent article.

Where connecting portion 200 comprises a discrete patch of material, such as discrete patch 250, patch 250 may be formed separately from absorbent assembly 44 and containment flaps 50, 52 and bonded to absorbent assembly 44 and containment flaps 50, 52. In such embodiments, patch 250 may be bonded to at least to containment flaps 50, 52. For instance, for a given embodiment, patch 250 may have a lateral dimension 280 such that, when placed on an absorbent article containing containment flaps 50, 52, patch 250 extends between containment flaps 50, 52 and encompasses at least a portion of each of containment flaps 50, 52. In some embodiments, patch 250 may have a lateral dimension 280 such that portions leg portions 260a, 260b and base portion 258 encompass the entire lateral extent of containment flaps 50, 52 (or at least the projection portions 66 of each of containment flaps 50, 52). In other embodiments, 250 may have a lateral dimension 280 such that portions leg portions 260a, 260b and base portion 258 encompass only a portion of the lateral extent of containment flaps 50, 52 (or at least the projection portions 66 of each of containment flaps 50, 52).

In any case, patch 250 may be bonded to containment flaps 50, 52 along bond regions 282 and 284, as seen in FIG. 6. In some exemplary embodiments, bond regions 282, 284 may align with projection portions 66 of containment flaps 50, 52. However, in other embodiments, bond regions 282, 284 may align with base portion 64 of containment flaps 50, 52. In still other embodiments, bond regions 282, 284 may align with bodyside liner 28, or another component disposed on the body facing surface 19 of absorbent article 10, 110, for instance in embodiments where containment flaps 50, 52 do not include base portion 64.

Bond regions 282, 284 may extend through both leg portions 260a, 260b and through base portion 258, as seen in FIG. 6. However, in other embodiments, bond regions 282, 284 may extend only through leg portions 260a, 260b. Additionally, although shown as generally rectangular, bond regions 282, 284 may form any suitable shape sufficient to adhere patch 250 to absorbent articles of the present disclosure, such as article 10 or 110.

In some additional embodiments where connecting portion 200 comprises a discrete patch, such as patch 250, patch 250 may be further bonded absorbent article 10 along bond region 286. Unlike bond regions 282, 284, bond 286 may bond patch 250 to absorbent article 10 along the bodyside liner 28, or along another component disposed on the body facing surface 19. Although, in other embodiments, instead of bond region 286 bonding patch 250 to the bodyside liner 28 or other component disposed on the body facing surface 19 of article 10, bond region 286 may bond patch 250 to an optional waistband. In any case, bonding patch 250 to absorbent article 10 along bond regions 282, 284, and 286 as described above may create a “pocket” to for BM exudates as they spread out along absorbent assembly 44 after passing through opening 201.

Although depicted as generally square in FIG. 6, bond region 286 may have any suitable shape including but not limited to circular, trapezoidal, rectangular, or the like. Additionally, bond region 286 may comprise multiple individual sub-bond regions. For example, patch 250 may comprise two bond regions 286 which are separated from each other and extend parallel to side edges 262 and 264. In this manner, patch 250 may form a pocket underneath tongue portion 256 after patch 250 has been bonded to the absorbent article as a middle portion of tongue portion 256 may be left un-bonded to the absorbent article. Bond region 286 may also extend into base portion 258 in some embodiments. In some further embodiments, bond region 286 may span across patch 250 and connect bond region 282 with bond region 284. In such embodiments, there may be only a single, continuous bond region, although the single region may generally conform to a shape similar to that depicted in FIG. 6.

Patch 250 may be bonded to the absorbent article along bond regions 282 and 284 (and possibly 286) using any bonding technique known in the art. For instance, patch 250 may be bonded to the absorbent article along bond regions 282 and 284 (and possibly 286) through application of adhesive, ultrasonic energy, heat energy, and/or pressure. In at least some embodiments, patch 250 may be have elastic properties—for instance, patch 250 may comprise elastic materials as described below—and may be bonded to the absorbent article in a stretched state. For instance, patch 250 may be stretched in its longitudinal dimension, e.g. in the direction of lengths 271, 272, and 273, before being applied to the article. In some embodiments, patch 250 may be stretched between about 110% and about 150% of its un-stretched length before being applied to the article. In other embodiments, patch 250 may be stretched between about 120% and about 130% of its un-stretched length before being applied to the article.

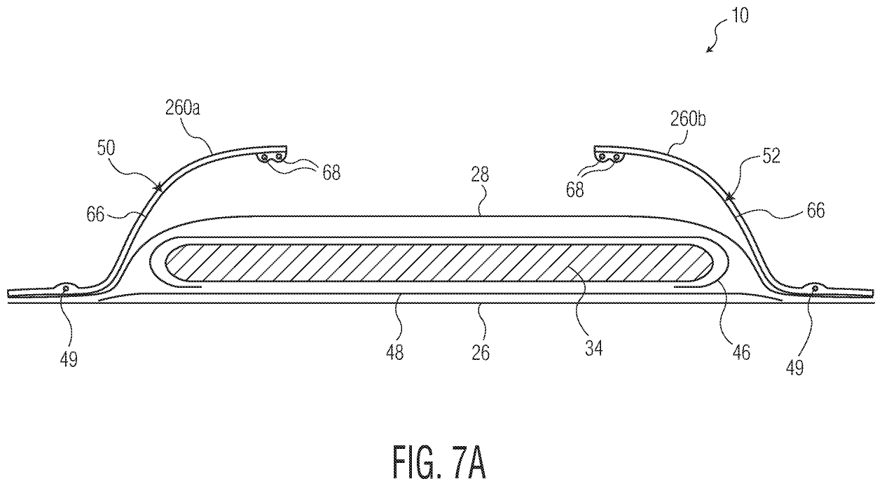

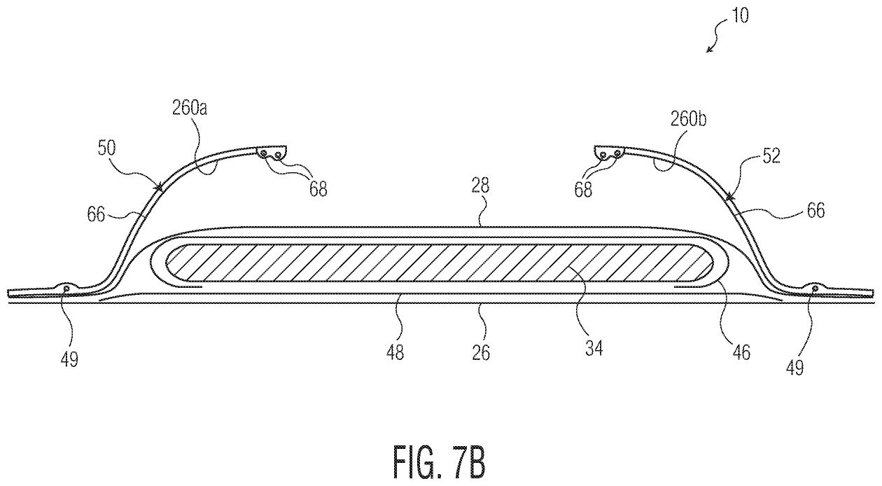

FIGS. 7A and 7B depict alternative contemplated embodiments for positioning of patch 250 in relation to containment flaps 50, 52. Specifically, FIGS. 7A and 7B depict an order of layers of absorbent article 10 as viewed along line 3-3 of FIG. 2. In the exemplary embodiment of FIG. 7A, leg portions 260a, 260b of patch 250 can be seen disposed on top of containment flaps 50, 52. Accordingly, in at least some embodiments, patch 250 may be bonded to absorbent article 10, 110 in a configuration where patch 250 is disposed over containment flaps 50, 52. FIG. 7B, depicts and an alternative embodiment. As can be seen, leg portions 260a, 260b are disposed underneath containment flaps 50, 52. Accordingly, in at least some embodiments, patch 250 may be bonded to absorbent article in a configuration where patch 250 is disposed underneath containment flaps 50, 52.

As mentioned, in some optional embodiments, absorbent articles of the present disclosure, such as articles 10, 110 may further include an elasticized waistband, such as waistband 202, located in the rear half sections 27, and more specifically in the rear waist region 14. In embodiments where connecting portion 200 of an absorbent article of the present disclosure comprises a patch, such as patch 250, and where the article further includes waist band 202, waistband 202 may cover at least a portion of patch 250, as seen in FIGS. 2-5. In some of these embodiments, waistband 202 may cover a portion of tongue portion 256. However, in other embodiments, waistband 202 may cover all of tongue portion 256. In still further embodiments, waistband 202 may cover all of tongue portion 256 and a portion of base portion 258. Additionally, as described previously, in at least some embodiments patch 250 may be bonded to bodyside liner 28 or another absorbent article component disposed on the body facing surface 19, for instance by bond region 286. However, in other embodiments, patch 250 may alternatively be bonded to waistband 202 through bond region 286 instead of to bodyside liner 28 or another component on body facing surface 19. In such embodiments, waistband 202 may be bonded to the absorbent articles of the present disclosure in a manner consistent with what is described in PCT/US15/23620, Attorney Docket Number 64967740PCT01 filed 31 Mar. 2015; PCT/US15/23596, Attorney Docket Number 64965789PCT01 filed 31 Mar. 2015; PCT/US15/23637, Attorney Docket Number 64967082PCT01 filed 31 Mar. 2015; PCT/US15/38271, Attorney Docket Number 64969698PCT01 filed 29 Jun. 2015; PCT/US15/47672, Attorney Docket Number 64970360PCT01 filed 31 Aug. 2015; and 62/212,051, Attorney Docket Number 64968524US01 filed 31 Aug. 2015. In such embodiments, patch 250 may increase the volume of the pocket formed by waistband 202, thereby providing a greater holding capacity for BM exudates and greater skin protection by covering a larger area of a wearer's skin.

In general, patch 250 may be constructed of any suitable material. Although, in at least some embodiments, patch 250 may comprise a material which is liquid impermeable so as to provide a barrier between BM exudates and a wearer's skin. Patch 250 may be a single piece construction comprising just a single layer of material, although this is not necessary in all embodiments. Patch 250 may instead have a multi-layer construction, for instance comprising two, three, four, or more layers. Again, in general, patch 250 may have any suitable construction and may be formed of any suitable material. As some examples, patch 250 may have the same construction and be formed of any of the materials described above with respect to outer cover 26, bodyside liner 28, or containment flaps 50, 52.

In at least some embodiments, patch 250 may comprise one or more elastic elements, thereby imparting elastic properties to patch 250. For instance, patch 250 may comprise one or more elastic strands, an elastic film, and/or an elastic laminate such as a stretch film laminate as described herein. Example suitable materials that may be used for patch 250 include vertical film laminate materials, stretch film laminate materials, or elastic laminate structures. One specific example material is the elastic film laminate material called SABBEEL herein, which is described in U.S. Pat. No. 8,287,677, titled “Printable Elastic Composite” and is expressly incorporated herein in its entirety. Additionally, it should be understood that these are only example materials.

FIGS. 8 and 9 depict further alternative embodiments of absorbent articles including connecting portions defining differently shaped openings than described previously with respect to absorbent article 10 and 110 of FIGS. 2-5. Specifically, FIG. 8 depicts exemplary absorbent article 310, including connecting portion 300 defining opening 301. As can be seen in FIG. 8, opening 301 is defined by laterally opposed edges 303 and 305. In the embodiment of FIG. 8, laterally opposed edges 303 and 305 connect such that there is no minimum separation distance between laterally opposed edges 303 and 305.

FIG. 9 depicts a further exemplary embodiment of an absorbent article, absorbent article 410 including connecting portion 400 defining opening 401. As can be seen in FIG. 9, opening 401 is defined by laterally opposed edges 403 and 405. In the embodiment of FIG. 9, the minimum separation distance 407 between laterally opposed edges 403 and 405 is at the maximum value described for minimum separation distance 207 detailed with respect to FIGS. 2-5. That is, minimum separation distance 407 equals the lateral distance between distal edges 465b of containment flaps 450, 452.

FIGS. 10-12 depict each of exemplary absorbent articles 10, 310, and 410, connecting portions 200, 300, and 400, and openings created by connecting portions 200, 300, and 400 as they would appear if worn by a wearer. FIGS. 10-12 were derived from absorbent articles configured according to FIGS. 2, 8, and 9 and viewed when placed on a clear test mannequin. FIG. 10 depicts absorbent article 10 including connecting portion 200 and opening 201. As can be seen opening 201 has become narrowed with respect to its depiction in FIG. 2 due to the in-use position of article 10. Narrowed opening 201 still has room for BM exudates to pass through opening 201 but is narrow enough to prevent the exudates from contacting a broad area of the wearer's skin as the exudates spread out on absorbent assembly 44 after passing through opening 201.

FIG. 11 depicts absorbent article 310 including connecting portion 300 and opening 301. As can be seen, opening 301 has also become narrowed with respect to its depiction in FIG. 8 due to the in-use position of article 310. However, opening 301 has narrowed so much that there is no effective opening for BM exudates to pass through past containment flaps of article 310 to contact the absorbent assembly of article 310.

FIG. 12 depicts absorbent assembly 410, including connecting portion 400 and opening 401. As can be seen, opening 401 has become narrowed with respect to its depiction in FIG. 9 due to the in-use position of article 410. However, opening 401 is still functional in that there is room for BM exudates to pass through the containment flaps of article 410 to contact the absorbent assembly of absorbent article 410. Although, opening 401 exposes a larger area of the wearer's skin to BM exudates after they pass through the containment flaps of the article 410 than the configuration according to FIG. 2. Accordingly, as can be see through FIGS. 8-13, careful selection of the shape of opening 201 is necessary to achieve a result of allowing BM exudates to pass beyond containment flaps to contact the absorbent assembly of an absorbent article while maintaining coverage over a relatively large area of a wearer's skin to protect the skin from the BM exudates as they spread over the absorbent assembly.

FIGS. 13 and 14 depict portions of an exemplary manufacturing processes for forming absorbent articles of the present disclosure, such as articles 10 and 110. In FIG. 10, depicts exemplary process 500 where discrete patches 550 are applied on top of containment flaps, resulting in a structure similar to that shown with respect to FIG. 7A. Process 500 comprises moving a continuous stream of absorbent assemblies 544 in machine direction 505. Absorbent assemblies 544 may include a chassis 511, including outer cover 526, bodyside liner 528, and absorbent core 534 sandwiched between outer cover 526 and bodyside liner 528. At the beginning of process 500, containment flaps 551, 552 may be brought into contact with and bonded to the continuous stream of absorbent assemblies 544. Containment flaps 551, 552 may be bonded to the continuous stream of absorbent assemblies 544 using suitable technique as known in the art. As one example, containment flaps 551, 552 may be bonded to the continuous stream of absorbent assemblies 544 using a barrier adhesive, such as described previously with respect to FIG. 3.

Once containment flaps 551, 552 have been bonded to the continuous stream of absorbent assemblies 544, the absorbent assemblies 544 and containment flaps 551, 552 may pass through adhesive applicator 520. Adhesive applicator 520 may apply adhesive onto containment flaps 551, 552, and in some embodiments to absorbent assemblies 544, in a predefined pattern. The predefined pattern may align with locations where discrete patches 550 will be applied to containment flaps 551, 552 and absorbent assemblies 544. For instance, the predefined pattern may align with bond regions of patches 550 as patches 550 are placed onto the containment flaps 551, 552 and the continuous stream of absorbent assemblies 544.

After passing adhesive applicator 520, the continuous stream of absorbent assemblies 544 including containment flaps 551, 552 may pass by transfer drum 535. Transfer drum may generally receive individual, separated patches 550 and transfer those individual, separated patches 550 onto the continuous stream of absorbent assemblies 544 and containment flaps 551, 552. As drum 535 rotates, patches 550 may come into contact with the continuous stream of absorbent assemblies 544 including containment flaps 551, 552, and more particularly the portions of the continuous stream of absorbent assemblies 544 including containment flaps 551, 552 where the adhesive has been applied by adhesive applicator 520 in the predefined pattern. Accordingly, as patches 550 contact the adhesive on the continuous stream of absorbent assemblies 544 including containment flaps 551, 552, patches 550 will adhere and bond to the continuous stream of absorbent assemblies 544 and containment flaps 551, 552, forming openings 561 as depicted in FIG. 13. In embodiments where patches 550 are applied in a pre-stretched state, the cut and transfer process depicted in FIG. 13 can further include a stretch or draw mechanism to impart elongation to the patches 550 before they are applied to the continuous stream of absorbent assemblies 544 and containment flaps 551, 552.

It should be understood that adhesive application is only one exemplary way in which to bond patches 550 to the continuous stream of absorbent assemblies 544 including containment flaps 551, 552. In other embodiments, ultrasonic energy, heat energy, and/or pressure, without the use of adhesive, may be used to bond patches 550 to the continuous stream of absorbent assemblies 544 including containment flaps 551, 552. In such embodiments, process 500 may not include adhesive applicator 520. Additionally, transfer drum 535 may additionally function as a portion of a rotary ultrasonic unit, e.g. as a pattern roll or an anvil roll. In such embodiments, patches 550 may still be bonded to the continuous stream of absorbent assemblies 544 including containment flaps 551, 552 along predefined bond regions, however the bonding may occur without the use of adhesives.

In some embodiments, patches 550 may be separated from a continuous length of patch material 545 before being applied to the continuous stream of absorbent assemblies 544 including containment flaps 551, 552. FIG. 13 depicts continuous length of patch material 545, including outlines 547 of individual patches 550. In actuality, outlines 547 may not be present in patch material 545, but are shown for ease of description.

Rollers 530, 531 may comprise a knife roll and an anvil roll. For instance, roller 530 may comprise a knife roll and roller 531 may comprise an anvil roll. Knife roll 530 may have one or more individual knife blades spaced circumferentially around roll 530, and each individual knife blade may have a shape that, when pressed again patch material 545, separates individual patches 550 from patch material 545 in a desired shape. For instance, the individual knife blades may have a shape that coincides with outlines 547. As individual patches 550 are cut from patch material at rollers 530, 531, the individual patches 550 may be captured on transfer roller 535, which transfers the patches 550 to the continuous stream of absorbent assemblies 544 including containment flaps 551, 552.

The speed of the continuous length of patch material 545, the rotational speed of rollers 530, 531, and the rotational speed of transfer drum 535 may be chosen such that individual patches 550 are separated from each other along this path so that they are applied to the continuous stream of absorbent assemblies 544 including containment flaps 551, 552 in an intermittent fashion, as shown in FIG. 13. Additionally, as seen in the embodiment of FIG. 13, patches 550 have a nested shape and are cut from patch material 545 in a manner where there is no wasted patch material 545. However, this does not have to be the case in all contemplated embodiments.

FIG. 14 depicts alternative exemplary process 600 where discrete patches 550 are first applied to the continuous stream of absorbent assemblies 544, and then the containment flaps 551, 557 are applied on top of patches 550 and the continuous stream of absorbent assemblies 544. Process 600 may result in a structure similar to that shown with respect to FIG. 7B.

In the alternative exemplary process 600, the continuous stream of absorbent assemblies 544 may first encounter adhesive applicator 520. In these embodiments, adhesive applicator 520 may also apply adhesive in a predefined to the absorbent assemblies 544. However, the predefined pattern in process 600 may be different than in process 500. For instance, in process 500, the predefined pattern may comprise adhesive being applied to containment flaps 551, 557 and possibly to the continuous stream of absorbent assemblies 544. In contrast, in process 600, the predefined pattern may only comprise adhesive being applied to the continuous stream of absorbent assemblies 544.

Next, similar to process 500, the continuous stream of absorbent assemblies 544 with applied adhesive encounters transfer drum 535, where individual patches 550 are applied to the continuous stream of absorbent assemblies 544. The patches 550 may be secured to the continuous stream of absorbent assemblies 544 by the adhesive applied by adhesive applicator 520. In some embodiments, the adhesive applied by adhesive applicator 520 may align with the tongue portions of patches 550. For instance, the applied adhesive may align with a bond region such as bond region 286 as described with respect to FIG. 6. In embodiments where patches 550 are applied in a pre-stretched state, the cut and transfer process depicted in FIG. 14 can further include a stretch or draw mechanism to impart elongation to the patches 550 before they are applied to the continuous stream of absorbent assemblies 544 and containment flaps 551, 557.

After passing transfer drum 535, the continuous stream of absorbent assemblies 544, now including patches 550, may pass through adhesive applicator 522. Like adhesive applicator 520, adhesive applicator 522 may also apply adhesive to the continuous stream of absorbent assemblies 544 (and including patches 550 in these embodiments) in a predefined pattern. In some embodiments, the predefined pattern may align with at least some portions of patches 550. For instance, the predefined pattern may encompass bond regions of patches 550, such as bond regions 282, 284 as described above with respect to FIG. 6. In some further embodiments, adhesive applicator 522 may also apply adhesive to secure containment flaps 551, 557 to the continuous stream of absorbent assemblies 544. For instance, adhesive applicator 522 may additionally apply barrier adhesive 49 as seen in FIG. 3.

After adhesive applicator 522, containment flaps 551, 557 may be applied to the continuous stream of absorbent assemblies 544 and patches 550. Accordingly, in the embodiment of FIG. 14, containment flaps 551, 557 are applied on top of patches 550, forming openings 561. In these embodiments, patches 550 end up positioned between bodyside liner 528 and containment flaps 551, 557.

In both of process 500 and 600, once containment flaps 551, 557 and patches 550 have been attached to the continuous stream of absorbent assemblies 544, individual absorbent articles may be formed from the continuous stream of absorbent assemblies 544. For instance, the continuous stream of absorbent assemblies 544 may continue on to a knife roll which separates the continuous stream of absorbent assemblies 544 into individual absorbent assemblies 544, a folding unit which folds the individual absorbent assemblies 544, and a packaging unit which packages the individual absorbent assemblies 544, as is known in the art.

Additionally, in at least some embodiments, additional components may be applied to the continuous stream of absorbent assemblies 544, or to the individual absorbent assemblies 544 after they are separated. For instance, as described, absorbent articles of the present disclosure may further comprise an elasticized waistband. Accordingly, in further embodiments of processes 500 and/or 600, an elasticized waistband may be applied to the continuous stream of absorbent assemblies 544 or separated, individual absorbent assemblies 544 over patches 550.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

Those skilled in the art will recognize that the present disclosure may be manifested in a variety of forms other than the specific embodiments described and contemplated herein. Specifically, the various features described with respect to the various embodiments and figures should not be construed to be applicable to only those embodiments and/or figures. Rather, each described feature may be combined with any other feature in various contemplated embodiments, either with or without any of the other features described in conjunction with those features. Accordingly, departure in form and detail may be made without departing from the scope of the present disclosure as described in the appended claims.

What is claimed is:

1. An absorbent article comprising:
a chassis extending in a longitudinal direction and a lateral direction, the chassis having a front half section with a front waist region including a front waist edge, a rear half section having a rear waist region including a rear waist edge, a body facing surface, and a garment facing surface;
an absorbent body; and
a pair of containment flaps extending between the front waist region and the rear waist region and attached to the body facing surface of the chassis, each of the pair of containment flaps having a flap distal edge, and the pair of containment flaps further including a connecting portion in the rear half section where the containment flaps and connecting portion define an opening to the body facing surface in the rear end section,
wherein the opening is defined by a pair of laterally opposed edges having a minimum separation distance within the rear end section of between about one-quarter and about three-quarters of a distance between the flap distal edges of the containment flaps.

2. The absorbent article of claim 1, wherein the pair of laterally opposed edges have a minimum separation distance between about one-third and about two-thirds of the distance between the flap distal edges of the containment flaps.

3. The absorbent article of claim 1, wherein:
the opening is further defined by a laterally extending back edge; and
the lateral extent of the laterally extending back edge comprises the minimum separation distance.

4. The absorbent article of claim 3, wherein each of the laterally opposed edges forms an angle with the laterally extending back edge of between about 60 degrees to about 85 degrees.

5. The absorbent article of claim 3, wherein each of the laterally opposed edges forms an angle with the laterally extending back edge of between about 65 degrees to about 75 degrees.

6. The absorbent article of claim 1, wherein the connecting portion comprises a web of material bonded to each of the pair of containment flaps.

7. An absorbent article comprising:
a chassis having a front half section with a front waist region including a front waist edge, a rear half section having a rear waist region including a rear waist edge, a body facing surface, and a garment facing surface;
an absorbent body;
a pair of containment flaps extending between the front waist region and the rear waist region and attached to the body facing surface of the chassis, each of the pair of containment flaps having a flap distal edge; and
a shaped patch bonded to the pair of containment flaps and the body facing surface of the absorbent article within the rear half section of the chassis,
wherein the shaped patch comprises a tongue portion, a base portion, and leg portions, and
wherein the leg portions are separated from each other by a minimum of between about one-quarter and about three-quarters of a distance between the flap distal edges of the containment flaps.

8. The absorbent article of claim 7, wherein the leg portions are separated from each other by a minimum of between about one-third and about two-thirds of a distance between the flap distal edges of the containment flaps.

9. The absorbent article of claim 7, wherein each of the leg portions forms an angle with the base portion of between about 60 degrees to about 85 degrees.

10. The absorbent article of claim 7, wherein each of the leg portions forms an angle with the base portion of between about 65 degrees to about 75 degrees.

11. The absorbent article of claim 7, wherein the shaped patch is positioned on top of each of the containment flaps.

12. The absorbent article of claim 7, wherein the shaped patch is positioned between the containment flaps and the body facing surface of the chassis.

13. The absorbent article of claim 7, further comprising an elastic waistband, and wherein the elastic waistband covers at least a portion of the tongue portion of the shaped patch.

14. The absorbent article of claim 7, wherein each of the leg portions are bonded to a respective containment flap, and wherein each of the leg portions extends laterally inward of the respective containment flap.

15. The absorbent article of claim 7, wherein at least a portion of the shaped patch is elasticized.

16. A method of forming an absorbent article comprising:
moving an absorbent article chassis containing an absorbent body in a machine direction, the absorbent article chassis comprising:
a longitudinal direction and a lateral direction;
a front half section with a front waist region including a front waist edge;
a rear half section having a rear waist region including a rear waist edge;
a body facing surface; and
a garment facing surface;
moving a pair of containment flaps in the machine direction, each of the containment flaps having distal flap edges;
bonding the pair of containment flaps to the absorbent article chassis at laterally opposed positions;
moving a length of shaped patch material in the machine direction;
separating a shaped patch from the shaped patch material, wherein the shaped patch comprises:
a tongue portion, a base portion, and leg portions, and
wherein the leg portions are separated from each other by a minimum of between about one-quarter and about three-quarters of a distance between the distal flap edges of the containment flaps after the containment flaps are bonded to the absorbent article chassis; and
bonding the shaped patch to the pair of containment flaps and to the body facing surface.

17. The method of claim 16, wherein the shaped patch is separated from the shaped patch material such that there is no waste of the shaped patch material.

18. The method of claim 16, wherein the shaped patch is bonded on top of the pair of containment flaps.

19. The method of claim 16, wherein the shaped patch is bonded between the pair of containment flaps and the garment facing surface.

20. The method of claim 16, further comprising bonding an elasticized waistband to the chassis of the absorbent article in the rear waist region and covering at least a portion of the shaped patch.
